(19) United States
(12) Patent Application Publication
(10) Pub. No.: US 2025/0108109 A1
(43) Pub. Date: Apr. 3, 2025

(54) FORMULATIONS COMPRISING FAB-PEG

(71) Applicants: UCB BIOPHARMA SRL, BRUSSELS (BE); BIOGEN MA INC., CAMBRIDGE, MA (US)

(72) Inventors: ANDREW JEFFREY YATES, SLOUGH, BERKSHIRE (GB); KATIA BENACCETTA, BRUSSELS (BE); JAN MASSANT, BRUSSELS (BE)

(21) Appl. No.: 18/715,243

(22) PCT Filed: Nov. 30, 2022
(86) PCT No.: PCT/EP2022/083929

§ 371 Date: May 31, 2024

(30) Foreign Application Priority Data
Dec. 1, 2021 (EP) 21211590.1

(51) Int. Cl. A61K 39/395; A61K 9/16; A61K 9/19; A61K 47/10; A61K 47/26; A61K 47/60

(52) U.S. Cl. CPC A61K 39/3955; A61K 9/1694; A61K 9/19; A61K 47/10; A61K 47/26; A61K 47/60

(57) ABSTRACT

The present invention relates to the field of pharmaceutical compositions. More particularly it is directed to pharmaceutical compositions comprising an antibody molecule, more particularly a Fab-PEG or a Fab′-PEG molecule, e.g., at high concentrations, and to methods of producing such formulations. Pharmaceutical compositions according to the invention can be lyophilised and are stable upon storage at a temperature from about 2 to 25° C. for an appropriate period of time.

### CROSS-REFERENCE TO RELATED APPLICATION

This application is the U.S. national stage application of International Patent Application No. PCT/EP2022/083929, filed Nov. 30, 2022.

### REFERENCE TO SEQUENCE LISTING

The Sequence Listing for this application is labeled “Seq-List.xml” which was created on Apr. 11, 2024 and is 13,784 bytes. The entire content of the sequence listing is incorporated herein by reference in its entirety.

### FIELD OF INVENTION

The invention relates to the field of pharmaceutical compositions. More particularly it is directed to pharmaceutical compositions comprising an antibody molecule, more particularly a Fab-PEG or a Fab′-PEG molecule, at high concentrations, and to methods of producing such pharmaceutical compositions. The pharmaceutical compositions according to the invention can also be lyophilised to provide freeze-dried formulations.

### BACKGROUND OF THE INVENTION

The industrial manufacture of therapeutic antibodies is a complex task. It requires scale-up from processes developed in a laboratory for producing antibodies in milligram quantities to multi-kilogram quantities. Despite antibodies being fairly stable molecules, when stored at high concentration over a period of time they may, nevertheless, suffer from chemical and physical instability. Typical chemical instability may result in deamidation, hydrolysis, oxidation, beta-elimination, disulphide exchange or reduction. Physical instability can result in denaturation, aggregation or precipitation.

Whilst liquid formulations can be administered with minimal preparation, they have the drawback of being less stable over time. The stabilization of proteins in liquid formulations to avoid or minimize aggregation, precipitation or degradation remains a particular challenge. Aggregation, with the formation of insoluble matter or precipitate, is a particular problem. As it may lead to immunological reactions upon administration and/or difficulty in performing proper administration of the pharmaceutical composition, e.g. by causing blockage of the delivery device.

Freeze-drying (i.e. lyophilisation) may be used for long term storage stabilization of antibodies that are otherwise unstable in liquid form. Freeze-drying antibodies at a lower temperature reduces the damage to the products and aids retaining molecular integrity. It extends the shelf life, reduces the temperature requirement for shipping and preserves the chemical and biological properties of the antibodies. Freeze-drying facilitates achieving high concentrations of the antibody in the final formulation, thus, reducing the injection volume, after reconstitution, as well as the injection time.

However, antibodies, as other proteins, undergo denaturation upon freeze-drying in the absence of stabilizers. Stabilizers may include certain sugars, polyols, amino acids, salts and polymers such as polyethylene glycol. Generally, a preferred stabilizer is chosen for a given formulation, although occasionally a combination of stabilizers may be used. It is generally thought that the mechanism of stabilization during drying is that the stabilizer acts as a water substitute. The interaction between water and proteins is thought to be important for the conformational stability of the proteins. When water is removed during drying, the stabilizers can form hydrogen bonds with the protein, as water molecules do, which may help to preserve the native protein structure during lyophilization process (Chang L, et al. 2005).

The level of stabilization afforded by sugars or polyols generally depends on their concentrations. Increasing sugar/polyol concentration to a certain level may eventually reach a limit of stabilization or even destabilize a protein during freeze-drying, so the particular ratio of stabilizer to protein may impact the storage stability of a freeze-dried antibody (Chang L, et al. 2005). A crystalline bulking agent may act as a filler increasing the density of the solid product and minimizing structure loss. It also typically provides homogeneous, dense compositions and it is generally easy to reconstitute. Glycine is another example of possible bulking agent in freeze-dried products where crystallizable compounds are necessary (Carpenter et al., 1997), to act by providing the appropriate texture so as to avoid apparent volume and consistency issues with the formulation such as collapse during the primary drying process. It has already been shown that glycine may further lead to a slight increase in stability in some circumstances (Meyer 2009), however contradicting data also exist (WO2007124090).

WO2016128318 and WO2017194646 describe, among various stabilizer combinations, formulations containing a full-length antibody, sucrose and glycine. U.S. Pat. No. 6,372,716 discloses a freeze-dried formulation containing factor IX in combination with glycine and sucrose.

WO2019096776 discloses spray-dried formulations containing a Fab-PEG molecule, a sugar (sucrose or trehalose) and at least one amino acid, such as glycine. However, the combination of sucrose and glycine did not show any promising results at least from a reconstitution viewpoint.

An important issue arising with a Fab-PEG or Fab′-PEG molecule is its stability with particular regard to hydrolysis and ring opening of the succinimide PEG linker which results in an increase in the acidic species levels.

Despite lyophilization being a good choice to achieve the desired long-term storage for antibody molecules, there is no simple, universal protocol in formulating antibody moieties such as Fab-PEG or Fab′-PEG molecule. These molecules can become unstable during lyophilization processes and/or long-term storage, aggregates may form upon reconstitution or lyophilized cakes may take too long to reconstitute. High concentration of stabilizers may affect the physico-chemical properties (i.e. viscosities) of the final formulation. Given the above, there remains a need in the art to provide further improved pharmaceutical compositions, more especially freeze-dried ones, comprising high concentrations of Fab-PEG or Fab′-PEG molecules.

### SUMMARY OF THE INVENTION

The present invention provides pharmaceutical compositions comprising a Fab-PEG or Fab′-PEG molecule at a concentration of from about 50 to about 200 mg/mL, a buffer keeping the pH between about 5.0 to about 7.0, from about 1.0 to about 5.0% w/v of sucrose, from about 0.5 to about 4.0% w/v of glycine, and optionally a surfactant. In some embodiments, the concentration of the buffer is at about 10 to 50 mM.

In some embodiments, pharmaceutical compositions according to the invention comprise 100 mg/ml of the Fab-PEG or Fab′-PEG molecule, about 20 mM of Histidine buffer which keep the pH at about 5.5, about 2.5% w/v of sucrose, about 2.0% w/v of glycine and optionally about 0.05% w/v of polysorbate 20.

Also provided herein are freeze-dried formulations obtained by freeze-drying any of the pharmaceutical compositions disclosed herein. In some embodiments, the present invention provides freeze-dried formulations comprising from about 50 to about 80% w/w of a Fab-PEG or Fab′-PEG molecule, from about 3 to about 14% w/w of a buffer keeping the pH between about 5.0 to about 7.0, from about 7 to about 30% w/w of sucrose, from about 3.5 to about 24% w/w of glycine, and optionally a surfactant.

In some embodiments, a Fab-PEG or Fab′-PEG molecule is derived from a humanised or human antibody. In some embodiments, a Fab-PEG or Fab′-PEG molecule specifically binds to CD40L.

The invention also provides methods for producing freeze-dried formulations comprising the steps of a. forming a mixture of a Fab-PEG or Fab′-PEG molecule, together with buffer, sucrose, glycine and optional surfactant to obtain a pharmaceutical composition; b. submitting the mixture of step a to freeze-drying and c. recovering the freeze-dried formulation, as well as a method for reconstituting the freeze-dried formulation according to the invention by adding a solvent.

Also described are articles of manufacture comprising a container comprising a pharmaceutical composition or a freeze-dried formulation according to the invention.

Pharmaceutical compositions and/or freeze-dried formulations herein described may be useful for administration in the treatment of a disease or disorder.

### Definitions

The term “comprising” as used herein does not exclude other elements, i.e. it is to be interpreted as encompassing all the specifically mentioned features as well as optional, additional, unspecified ones.
Where an indefinite or definite article is used when referring to a singular noun, e.g. “a”, “an” or “the”, this includes a plural of that noun unless something else is specifically stated.
The terms “about” or “approximately” denote an interval of accuracy that the person skilled in the art will understand to still ensure the technical effect of the feature in question. The term typically indicates deviation from the indicated numerical value of ±10%, and preferably of ±5%. Where the term “about” is used the application also discloses employing the exact value specified. Where point values are referred to, the application also discloses about such values being employed, the same being the case for endpoints of ranges. For example should the description specify “about 50 to about 200”, that would mean “50±10% to 200±10%” as well as “50 to 200”.
The term “CD40L” or “CD40 ligand”, refers to the protein also known as CD154, CD40 counter receptor (CD40CR), gp39, T-BAM, T-Cell Activating Molecule, TRAF, TNF-Related Activation Protein (TRAP), and Tumor Necrosis Factor Ligand Superfamily Member 5 (TNFSF5). It is a 39 kDa type II membrane glycoprotein of the TNF family. CD40L is a polypeptide of 261 amino acids, consisting of a 215 amino acids extracellular domain, a 24 amino acids transmembrane region, and a 22 amino acids cytoplasmic tail.
The term “antibody” or “antibodies” as used herein refers to monoclonal antibodies and is not limited to recombinant antibodies that are generated by recombinant technologies as known in the art. “Antibody” or “antibodies” include antibodies of any species, in particular of mammalian species, having two essentially complete heavy and two essentially complete light chains, human antibodies of any isotype, including IgA1, IgA2, IgD, IgG1, IgG2a, IgG2b, IgG3, IgG4, IgE and IgM, modified variants thereof, and/or antigen-binding fragments thereof.
The term “Fab” as used herein refers to an antibody fragment comprising one constant domain and one variable domain of a heavy chain as well as one constant domain and one variable domain of a light chain of an antibody. A “Fab” molecule is a Fab further comprising the hinge domain.
The term “Fab-PEG” or “Fab′-PEG” relates to a molecule comprising respectively a Fab or Fab′ moiety combined to a PEG moiety. Fab or Fab′ molecules can contain a modified amino acid residue, e.g., a cysteine or lysine residue, in order to be pegylated. For example, a Fab molecule may be a modified Fab fragment wherein the modification is the addition to the C-terminal end of its heavy chain one or more amino acids to allow the attachment of an effector molecule. The additional amino acids may form a modified hinge region containing one or more cysteine residues to which the effector molecule may be attached.

A Fab or Fab′ moiety can be obtained by culturing prokaryotic or eukaryotic host cells transfected with one or more expression vectors encoding the recombinant Fab or Fab′ moiety. For example, the eukaryotic host cells may be mammalian cells, such as Chinese Hamster Ovary (CHO) cells. Prokaryotic host cells are typically gram-negative bacteria, such as E. coli cells. Host cells may be cultured in any medium that will support their growth and expression of the recombinant protein. The best conditions for each host cell are known to those skilled in the art.

Once recovered either from the supernatant of a cell culture, from the periplasmic space or from inclusion bodies, depending on the host cell used for the production, a Fab or Fab′ moiety can be purified. Purification methods are well-known to those skilled in the art. Purification methods typically comprise a combination of various chromatographic and filtration steps. The full process is typically performed in aqueous condition. After a first series of purification steps, a Fab or Fab′ moiety can be pegylated and further purified. The solution recovered at the end of the process can be submitted to formulation.
The term “PEG” refers to poly(ethyleneglycol) moieties. Other appropriate PEG compounds include, but are not limited to, maleimido monomethoxy PEG, activated PEG polypropylene glycol, methoxypoly(ethyleneglycol) polymer, but also charged or neutral polymers of the following types: dextran, colominic acids, or other carbohydrate-based polymers, polymers of amino acids, and biotin and other affinity reagent derivatives. The size of the PEG compound may be varied as desired but will generally be in an average molecular weight range from 500 Da to 50000 Da, for example from 5000 to 40000 Da such as from 20000 to 40000 Da. The polymer size may in particular be selected on the basis of the intended use of the product for example ability to localize to certain tissues such as tumours or extend circulating half-life (see Chapman, 2002). Thus, for example, where the product is intended to leave the circulation and penetrate tissue, for example for use in the treatment of a tumour, it may be advantageous to use a small molecular weight polymer, for example with a molecular weight of around 5000 Da. For applications where the product remains in the circulation, it may be advantageous to use a higher molecular weight polymer, for example having a molecular weight in the range from 20000 Da to 40000 Da.
The term “PEGylation” refers to the attachment, e.g. through covalent bonding, of a PEG moiety or a derivative thereof to its partner molecule (such as a Fab or Fab′ molecule), with or without coupling agents or derivatization with coupling or activating moieties (e.g., with thiol, triflate, tresylate, aziridine, oxirane, and/or a maleimide moiety, e.g., PEG-maleimide). For example, a PEG moiety may be attached to a cysteine in the hinge region of a Fab′ or a modified Fab. Alternatively or additionally, a PEG modified Fab fragment may have a maleimide group covalently linked to a single thiol group in a modified hinge region. A lysine residue may be covalently linked to the maleimide group and to each of the amine groups on the lysine residue may be attached a methoxypoly(ethyleneglycol) polymer having a molecular weight of approximately 20,000 Da. The total molecular weight of the PEG attached to the Fab fragment may therefore be approximately 40,000 Da. The best pegylation methods are well-known to those skilled in the art.
The term “specifically binds to CD40L”, “specifically binding to human CD40L”, and equivalents as used herein means the Fab or Fab′ molecule, pegylated or not, will bind to CD40L/human CD40L with sufficient affinity and specificity to achieve a biologically meaningful effect. The selected molecule will normally have a binding affinity for CD40L/human CD40L, for example, the Fab or the Fab′ molecule may bind human CD40L with a Kd value of between 100 nM and 1 pM. The term “Kd”, as used herein, is intended to refer to the dissociation constant of a particular Fab or Fab′ molecule-antigen interaction, representing the Fab or Fab′ molecule affinity to its target. Affinities may be determined for example by a surface plasmon resonance assay, such as the BIAcore assay; enzyme-linked immunoabsorbent assay (ELISA); and/or competition assays (e.g. RIA's). Within the meaning of the present invention a Fab or Fab′ molecule specifically binding to CD40L/human CD40L may also bind to another molecule, e.g. cyno CD40L. The Fab or Fab′ molecule binding specifically to CD40L/human CD40L may also neutralizes CD40L/human CD40L.
The term “neutralizes” as used herein refers to a Fab or Fab′ molecule, pegylated or not, that inhibits or substantially reduces the biological effect of the molecule to which it specifically binds. Therefore, the expression “the Fab or Fab′ molecule neutralizes CD40L/human CD40L” refers to a Fab or Fab′ molecule that specifically binds to CD40L or human CD40L and inhibits or substantially reduces the biological effect thereof such as by blocking CD40L binding to CD40.
The term “high concentration”, or related terms, in relation to the Fab or Fab′ molecule, pegylated or not, means that the concentration of said molecule is at least 50 mg/mL.
The term “stability”, as used herein, refers to the physical, chemical, and conformational stability of a Fab or Fab′ molecule, pegylated or not, in the formulations according to the present invention. Instability of a protein formulation may be caused for instance by chemical degradation or aggregation of the protein molecules to form higher order polymers, deglycosylation, modification of glycosylation or oxidation.
The term “stable formulation” refers to a formulation in which the protein of interest (here a Fab or Fab′ molecule, pegylated or not) essentially retains its physical, chemical and/or biological properties upon storage. In order to measure the protein stability in a formulation, various analytical methods are well within the knowledge of the skilled person (see some examples in the example section). Stability is typically assessed at a selected temperature (for instance −70° C., 2-8° C., 25° C., 35° C. or more) for a selected time period (e.g. 3 months, 6 months, 12 months or more). As a Fab or Fab′ molecule (pegylated or not), once formulated, is typically stored in the fridge (typically 2-8° C.) or at room temperature (typically 15-25° C.) before being administered to a patient, it is important that said formulated molecule is stable over time at least at 2-25° C., as shown for example at 2-8° C. and 25° C. Various values can be used to characterise stability over a given time period (in comparison with the initial data), such as (and not limited to): 1) no more than 10% of alteration per year of the monomeric form of the antibody when the storage is performed at 25° C., 2) an alteration of no more than about 0.5 point/year (0.5% per year) of High Molecular Weight Species (HMW or HMWS; also herein referred to as aggregates) when the storage is performed at 2-8° C. or no more than about 3 points/year (3% per year) of HMWS when the storage is performed at 25° C., and/or 3) no more than +/−0.3 unit variation of the pH.
As used herein, “pharmaceutical composition” can also be referred to as “stable pharmaceutical composition”, “formulation” or “stable formulation” without any differentiation.
The term “buffer”, as used herein, refers to solutions of compounds that are known to be safe in formulations for pharmaceutical or veterinary use and that have the effect of maintaining or controlling the pH of the formulation in the pH range desired for the formulation.
The term “surfactant”, as used herein, refers to a soluble compound that can be used notably to increase the water solubility of hydrophobic, oily substances or otherwise increase the miscibility of two substances with different hydrophobicities. For this reason, these polymers are commonly used in industrial applications, cosmetics, and pharmaceuticals. They are also used as model systems for drug delivery applications, notably in order to modify the absorption of the drug or its delivery to the target tissues. Well known surfactants include polysorbates (polyoxyethylene derivatives, also known as Tween; such as PS20 or PS80) as well as poloxamers (i.e. copolymers based on ethylene oxide and propylene oxide, also known as Pluronics®).
The term “stabilizing agent”, “stabilizer” or “isotonicity agent”, as used herein, is a compound that is physiologically tolerated and imparts a suitable stability/tonicity to a formulation. It prevents notably the net flow of water across cell membranes that are in contact with the formulation. During freeze-drying (lyophilisation) process, the stabilizer is also effective as a cryoprotectant. Compounds such as glycerin, are commonly used for such purposes. Other suitable stability agents include, but are not limited to, amino acids or proteins, salts (e.g. sodium chloride), and sugars (e.g. dextrose, trehalose, sucrose and lactose). In some embodiments, the stabilizing agent is sucrose.
The term “bulking agent” refers to a chemical entity which provides structure to the lyophilizate or residual solid mass of a pharmaceutical preparation after it has been lyophilized and which protects it against collapse. A crystallizable bulking agent shall mean a bulking agent as described herein which can be crystallized during lyophilization. Said bulking agent can for instance be glycine.
The term “freeze-drying” also known as “lyophilization” refers to a process for obtaining a freeze-dried formulation consisting of at least three main steps: 1) lowering the temperature of the product to be freeze-dried to below freezing point (typically between −40 and −80° C.; freezing step), 2) high-pressure vacuum (typically between 30 and 300 mTorr; first drying step) and 3) increasing the temperature (typically between 2° and 40° C.; second drying step). Techniques for the lyophilization of solutions comprising antibodies are well known in the art. Many commercially available monoclonal antibody products such as SYNAGIST, REMICADE™, CIMZIA®, RAPTIVA™, SIMULECTT, XOLAIR™ and HERCEPTIN™ are supplied as a freeze-dried formulation. Freeze-dried formulations are also known as “lyophilised formulations” or yet “lyophilizates”.
The term “vial” or “container”, as used herein, refers broadly to a reservoir suitable for retaining formulations described herein in a liquid form or a lyophilized form. Similarly, it will retain the solvent for reconstitution should the formulation be under a lyophilized form. Examples of a vial that can be used in the present invention include an ampoule, a tube, a bottle, a syringe (such as a pre-filled syringe), cartridges, or other such reservoir suitable for delivery of the formulation to the patient via injection, for example, via intravenous infusion or subcutaneous injection.
The term “solvent”, as used herein, refers to an aqueous solvent. Said aqueous solvent may consist solely of water, or may comprise water plus one or more miscible solvents, and may contain dissolved solutes such as sugars, buffers, salts or other excipients. Non limiting aqueous solvents include water (such as water for injection) or yet saline solvent or saline buffer (such as PBS).
The term “reconstitution time” as used herein means the time it takes to reconstitute a lyophilizate in a desired volume of solvent (e.g. water or saline buffer). Unexpectedly superior excipients reduce the overall reconstitution time of at least about 10% or more. Reconstitution time depends on the final volume of reconstitution reconstituted. Typically, for instance, acceptable reconstitution times are considered to be within 10 minutes or less for a volume of 1 mL.

### DETAILED DESCRIPTION OF THE INVENTION

The invention is based, in part, on the surprising finding from the inventors that the combination of sucrose and glycine, more particularly at certain ratios, in a buffered solution leads to Fab-PEG or Fab′-PEG molecule-containing freeze-dried pharmaceutical compositions providing long-term stability to said Fab-PEG or Fab′-PEG molecule, without affecting the processability of the pharmaceutical composition. It is a finding from the inventors that the pharmaceutical compositions according to the invention are stable over time, in particular when stored in a lyophilised state, at 2-25° C., as shown for example at 2-8° C. and 25° C. In some embodiments, freeze-dried formulations according to the invention are also easy to reconstitute (less than 20 min).

In one aspect, the present invention provides a liquid pharmaceutical composition comprising:
a. a Fab-PEG or Fab′-PEG molecule at a concentration of from about 50 to about 200 mg/mL,
b. a buffer keeping the pH between about 5.0 to about 7.0,
c. from about 1 to about 5% w/v of sucrose,
d. from about 0.5 to about 4% w/v of glycine, and
e. optionally a surfactant.

In some embodiments, the Fab-PEG or Fab′-PEG molecule is preferably derived from a humanised or human antibody which preferably specifically binds to CD40L, such as human CD40L.

In some embodiments, said Fab-PEG or Fab′-PEG molecule is a pegylated Fab or Fab′ fragment disclosed in WO 2008/118356 (incorporated herein in its entirety) and has a light chain variable region (LCVR) with the CDR1, CDR2 and CDR3 having the amino acid sequence of SEQ ID NOs: 4, 5 and 6, respectively, and a heavy chain variable region (HCVR) with the CDR1, CDR2 and CDR3 having the amino acid sequence of SEQ ID NOs: 1, 2 and 3, respectively. In some embodiments, said Fab-PEG or Fab′-PEG molecule has the VL chain sequence shown in SEQ ID NO: 7 and the VH chain sequence shown in SEQ ID NO: 8. In some embodiments, said Fab-PEG or Fab′-PEG molecule has a light chain sequence as shown in SEQ ID NO: 9 and a heavy chain sequence as shown in SEQ ID NO: 10. In some embodiments, said Fab-PEG or Fab′-PEG molecule is PEGylated at a cysteine in the modified hinge region as described in WO 2008/118356. In some embodiments, a maleimide group is covalently linked to a single thiol group in a cysteine in the modified hinge region. In some embodiments, a lysine residue is then covalently linked to said maleimide group and a methoxypoly(ethyleneglycol) polymer having a molecular weight of approximately 20 KDa is attached to each of the amine groups on the lysine residue. The total molecular weight of the entire PEG covalently linked to the monovalent Fab′ may therefore be approximately 40 KDa. An anti-CD40L Fab-PEG or Fab′-PEG molecule according to the invention may also comprise a light chain variable region comprising SEQ ID NO: 7 or sequences at least 80% identical thereto, and/or a heavy chain variable region comprising SEQ ID NO: 8 or sequences at least 80% identical thereto. Alternatively or additionally, an anti-CD40L Fab-PEG or Fab′-PEG molecule according to the invention may also comprise a light chain comprising SEQ ID NO: 9 or sequences at least 80% identical thereto, and/or a heavy chain comprising SEQ ID NO: 10 or sequences at least 80% identical thereto.

Therefore, in some embodiments, pharmaceutical compositions and/or freeze-dried formulations according to the invention comprise a Fab-PEG or Fab′-PEG molecule which:
a. comprises CDR-H1 having the sequence as defined in SEQ ID NO:1; CDR-H2 having the sequence as defined in SEQ ID NO:2; CDR-H3 having the sequence as defined in SEQ ID NO:3; CDR-L1 having the sequence as defined in SEQ ID NO:4; CDR-L2 having the sequence as defined in SEQ ID NO:5 and CDR-L3 having the sequence as defined in SEQ ID NO:6; or
b. comprises a light variable region having the sequence as defined in SEQ ID NO: 7 and a heavy variable region having the sequence as defined in SEQ ID NO: 8; or
c. comprises a light variable region having at least 80% identity, preferably 90% identity to the sequence as defined in SEQ ID NO: 7 and a heavy variable region having at least 80% identity, preferably 90% identity to the sequence as defined in SEQ ID NO: 8.

Throughout this specification, complementarity determining regions (“CDR”) are defined according to the Kabat definition (Kabat et al., (1991), 5th edition, NIH publication No. 91-3242).

In some embodiments, a Fab-PEG or Fab′-PEG molecule is present in the pharmaceutical composition at a concentration of about 50 to about 200 mg/mL, for example, between about 70 to about 150 mg/mL, such as about 70, about 75, about 80, about 85, about 90, about 95, about 100, about 105, about 110, about 115, about 120, about 125, about 130, about 135, about 140, about 145, or about 150 mg/mL. Alternatively or additionally, a Fab-PEG or Fab′-PEG molecule is present in a liquid pharmaceutical composition in an amount expressed in terms of weight (grams) per 100 mL (% w/v). Thus, in some embodiments, a Fab-PEG or Fab′-PEG molecule present in the pharmaceutical composition as a whole can be present in an amount of about 5 to about 20% w/v, for example, of about 7 to about 15% w/v, such as about 7.0, about 7.5, about 8.0, about 8.5, about 9.0, about 9.5, about 10.0, about 10.5, about 11.0, about 11.5, about 12.0, about 12.5, about 13.0, about 13.5, about 14.0, about 14.5, or about 15.0% w/v. Once a pharmaceutical composition is lyophilised, the Fab-PEG or Fab′-PEG molecule is present in an amount that may be expressed in terms of weight per weight (% w/w). Thus, in some embodiments, a Fab-PEG or Fab′-PEG molecule present in a lyophilizate (i.e. freeze-dried formulation) as a whole can be present in an amount of about 50 to about 80% w/w, for example, of about 55 to about 75% w/w, such as of about 55, about 56, about 57, about 58, about 59, about 60, about 61, about 62, about 63, about 64, about 65, about 66, about 67, about 68, about 69, about 70, about 71, about 72, about 73, about 74 or about 75.0% w/w. For example, in some embodiments, the concentration of a Fab-PEG or Fab′-PEG molecule in a lyophilizate is about 67.0% w/w.

Liquid pharmaceutical compositions according to the invention as a whole comprise a buffer keeping the pH between about 5.0 to about 7.0. Acceptable buffers for controlling pH at a moderately acidic pH to a neutral pH include, but are not limited to, phosphate, acetate, citrate, histidine, arginine, TRIS, and histidine buffers. “TRIS” refers to 2-amino-2-hydroxymethyl-1,3,-propanediol, and to any pharmacologically acceptable salt thereof. In some embodiments, liquid pharmaceutical compositions comprise a histidine buffer, such as a histidine-HCl buffer, keeping the pH comprised between 5.0 (or about 5.0) and 7.0 (or about 7.0), for example comprised between 5.0 (about 5.0) and 6.0 (about 6.0), such as (about) 5.0, 5.1, 5.2, 5.3, 5.4, 5.5, 5.6, 5.7, 5.8, 5.9 or 6.0. In all the embodiments of the present invention, unless otherwise indicated, the pH value was measured at 23-25° C. and it is within ±0.1 or ±0.2 of a pH unit. In some embodiments a histidine buffer is a buffer comprising a mixture of histidine and histidine-HCl whose proportions are determined by the desired pH as determined by the skilled artisan, and may be referred to herein also as histidine/histidine-HCl buffer.

In some embodiments, buffer concentration in a liquid pharmaceutical composition is at about 10 to about 100 mM. In some embodiments, the concentration of the buffer is at about 10 to about 50 mM, such as at about 10, about 15, about 20, about 25, about 30, about 35, about 40, about 45 or about 50 mM. For example, the concentration of the buffer can be at or at about 20 mM. Once the pharmaceutical composition is lyophilised, the buffer is present in an amount that may be expressed in terms of weight per weight (% w/w). In such a case, the lyophilizate comprises buffer present in an amount of about 2% to about 14% w/w or of about 3 to about 14% w/w, for example of about 2% to about 12% w/w or to 3 to about 12% w/w, such as of about 2, about 3, about 4, about 5, about 6, about 7, about 8, about 9, about 10, about 11 or about 12% w/w. For example, in some embodiments, the concentration of the buffer in the lyophilizate is about 2.5-2.6% w/w. In further embodiments, the concentration of the buffer in the lyophilizate is about 2.6-2.8% w/w.

In the context of the invention as a whole, sucrose is present in a liquid pharmaceutical composition in an amount of about 1 to about 5% w/v, for example from about 1.5 to about 4% w/v, or in some embodiments, in an amount of about 2 to about 4% w/v such as in an amount of about 2.0, about 2.25, about 2.5, about 2.75, about 3.0, about 3.25, about 3.5, about 3.75 or about 4.0% w/v. For example, the amount of sucrose can be at about 2.5% w/v. Once the pharmaceutical composition is lyophilised, sucrose is present in an amount that may be expressed in terms of weight per weight (% w/w). In such a case, the lyophilizate comprises sucrose in an amount of about 7 to about 30% w/w, for example of about 10 to about 25% w/w, or in some embodiments, of about 15 to 25% w/w, such as of about 15, about 16, about 17, about 18, about 19, about 20, about 21, about 22, about 23, about 24 or about 25% w/w. For example, in some embodiments, the amount of sucrose in the lyophilizate can be at about 16.5-16.75% w/w.

In the context of the invention as a whole, glycine is present in a liquid pharmaceutical composition in an amount of about 0.5 to about 4% w/v, for example from about 1 to about 3% w/v, or in an amount of about 1.5 to about 3% w/v such as in an amount of about 1.5, about 1.75, about 2.0, about 2.25, about 2.5, about 2.75, about 3.0% w/v. In some embodiments, glycine that is used according to the present invention is L-glycine. For example, the amount of glycine can be at about 2.0% w/v. Once the pharmaceutical composition is lyophilised, glycine is present in an amount that may be expressed in terms of weight per weight (% w/w). In such a case, the lyophilizate comprises glycine in an amount of about 3.5 to about 24% w/w, for example of about 7 to about 20% w/w, or of about 10 to about 20% w/w, such as of about 10, about 11, about 12, about 13, about 14, about 15, about 16, about 17, about 18, about 19 or about 20% w/w. For example, in some embodiments, the amount of glycine in the lyophilizate can be at about 13.3-13.5% w/w.

In the context of the disclosure as a whole, a surfactant may optionally be present in the liquid pharmaceutical composition. When present, said surfactant is for example a polysorbate such as PS20. The surfactant is added in the aqueous antibody molecule-containing solution at a concentration of about 0.01% to about 0.2% w/v, for example, of about 0.02% to about 0.1% (w/v), such as of about 0.02, about 0.03, about 0.04, about 0.05, about 0.06, about 0.07, about 0.08, about 0.09 or about 0.1% (w/v). For example, the surfactant is PS20 and is in an amount of about 0.05% w/v. Once the pharmaceutical composition is lyophilised, the surfactant is present in an amount expressed in terms of weight per weight (% w/w). In such a case, the lyophilizate comprises a surfactant in an amount of about 0.07 to 1.3% w/w, for example of about 0.1 to 0.7% w/w, such as of about 0.1, about 0.2, about 0.3, about 0.4, about 0.5, about 0.6 or about 0.7% w/w. For example, the surfactant is PS20 and its amount in the lyophilizate can be of or of about 0.3% w/w.

It has been surprisingly shown by the inventor, in the context of the invention as a whole, that the ratio weight/weight (w/w) sucrose:glycine plays a key role in the performances of lyophilizates. In some embodiments, the ratio (w/w) sucrose:glycine is from about 2:3 to about 2:1. In some embodiments, the ratio (w/w) sucrose:glycine is from about 3:4 to about 5:3 or from about 4:5 to about 3:2. In some embodiments, the ratio (w/w) sucrose:glycine can be about 3:4, about 4:5, about 5:6, about 9:10, about 11:12, about 1:1, about 13:12, about 7:6, about 6:5, about 5:4 or about 4:3. For example, the ratio (w/w) sucrose:glycine can be about 1:1 or about 5:4. Therefore, in some embodiments, herein provided are liquid pharmaceutical compositions comprising:
a. a Fab-PEG or Fab′-PEG molecule at a concentration of from about 50 to about 200 mg/mL,
b. a buffer keeping the pH between about 5.0 to about 7.0,
c. sucrose and glycine in a ratio (w/w) sucrose:glycine of about 2:3 to about 2:1, and
d. optionally a surfactant.

In some embodiments, a liquid pharmaceutical composition comprises or consists of about 50 to about 200 mg/ml of a Fab-PEG or Fab′-PEG molecule, about 10 to about 100 mM of a buffer (such as an histidine buffer) at pH of about 5.0 or about 7.0, about 1 to about 5% w/v of sucrose, about 0.5 to about 4% w/v of glycine and optionally about 0.01 to about 0.2% w/v of surfactant (such as PS20). In some embodiments, a liquid pharmaceutical composition comprises or consists of about 70 to about 150 mg/ml of a Fab-PEG or Fab′-PEG molecule, about 10 to about 50 mM of a buffer (such as an histidine buffer) at pH of about 5.0 to about 6.0, about 1.5 to about 4% w/v of sucrose, about 1 to about 3% w/v of glycine and optionally about 0.02 to about 0.1% w/v of surfactant (such as PS20). In some embodiments, the liquid pharmaceutical composition of the present invention comprises or consists of a Fab-PEG or Fab′-PEG molecule at about 70 to about 150 mg/mL, about 10 to about 50 mM of a buffer (such as an histidine buffer) at pH of about 5.0 to about 6.0, about 2 to about 4% w/v of sucrose, about 1.5 to about 3% w/v of glycine and optionally about 0.02 to about 0.1% w/v of surfactant (such as PS20). For instance, a liquid pharmaceutical composition according to the invention can comprise about 100 mg/ml of a Fab-PEG or Fab′-PEG molecule, about 20 mM of Histidine buffer which keep the pH at about 5.5, about 2.5% w/v of sucrose, about 2% w/v of glycine and about 0.05% w/v of polysorbate 20. In some embodiments, a liquid pharmaceutical composition according to the invention can comprise about 100 mg/ml of a Fab-PEG or Fab′-PEG molecule, about 20 mM of Histidine buffer which keep the pH at about 5.5, about 2.5% w/v of sucrose and about 2% w/v of glycine. In some embodiments, a liquid pharmaceutical composition comprises or consists of about 50 to about 200 mg/mL of a Fab-PEG or Fab′-PEG molecule, about 10 to about 100 mM of a buffer (such as an histidine buffer) at pH of about 5.0 or about 7.0, sucrose and glycine in a respective ratio (w/w) of about 2:3 to about 2:1 and optionally about 0.01 to about 0.2% w/v of surfactant (such as PS20). In some embodiments, a liquid pharmaceutical composition comprises or consists of about 70 to about 150 mg/ml of a Fab-PEG or Fab′-PEG molecule, about 10 to about 50 mM of a buffer (such as an histidine buffer) at pH of about 5.0 to about 6.0, sucrose and glycine in a respective ratio (w/w) of about 3:4 to about 5:3 and optionally about 0.02 to about 0.1% w/v of surfactant (such as PS20). In some embodiments, the liquid pharmaceutical composition of the present invention comprises or consists of a Fab-PEG or Fab′-PEG molecule at about 70 to about 150 mg/mL, about 10 to about 50 mM of a buffer (such as an histidine buffer) at pH of about 5.0 to about 6.0, sucrose and glycine in a respective ratio (w/w) of about 4:5 to about 3:2 and optionally about 0.02 to about 0.1% w/v of surfactant (such as PS20). For instance, a liquid pharmaceutical composition according to the invention can comprise about 100 mg/mL of a Fab-PEG or Fab′-PEG molecule, about 20 mM of Histidine buffer which keep the pH at about 5.5, sucrose and glycine in a respective ratio (w/w) of 1:1 or about 5:4 and about 0.05% w/v of polysorbate 20. In some embodiments, a liquid pharmaceutical composition according to the invention can comprise about 100 mg/mL of a Fab-PEG or Fab′-PEG molecule, about 20 mM of Histidine buffer which keep the pH at about 5.5 and sucrose and glycine in a respective ratio (w/w) of about 1:1 or about 5:4. The present invention further describes freeze-dried formulations. Techniques for the lyophilization of liquid pharmaceutical compositions comprising a Fab-PEG or Fab′-PEG molecule are well known in the art.

The present invention further describes a freeze-dried formulation comprising from about 50 to about 80% w/w of a Fab-PEG or Fab′-PEG molecule, from about 2% to about 12% w/w or from about 3 to about 14% w/w of a buffer keeping the pH between about 5.0 to about 7.0, from about 7 to about 30% w/w of sucrose, from about 3.5 to about 24% w/w of glycine, and optionally a surfactant. In some embodiments, a freeze-dried formulation (i.e. the lyophilizate) comprises from about 58 to about 75% w/w of a Fab-PEG or Fab′-PEG molecule, from about 2% to about 12% w/w or from about 3 to about 12% w/w of a buffer keeping the pH between about 5.0 to about 7.0, from about 10 to about 25% w/w of sucrose, from about 7 to about 20% w/w of glycine, and optionally from about 0.07 to about 1.3% w/w of a surfactant. In some embodiments, a lyophilizate comprises from about 58 to about 75% w/w of a Fab-PEG or Fab′-PEG molecule, from about 2% to about 12% w/w or from about 3 to about 12% w/w of a buffer keeping the pH between about 5.0 to about 7.0, from about 15 to about 25% w/w of sucrose, from about 10 to about 20% w/w of glycine, and optionally from about 0.1 to about 0.7% w/w of a surfactant. In some embodiments, a lyophilizate comprises about 67% w/w of a Fab-PEG or Fab′-PEG molecule, about 2.5-2.6% w/w of a buffer keeping the pH between about 5.0 to about 7.0, about 16.5-16.75% w/w of sucrose, about 13.3-13.5% w/w of glycine, and optionally about 0.3% w/w of a surfactant. In a further embodiment, the freeze-dried formulation comprises about 67% w/w of a Fab-PEG or Fab′-PEG molecule, about 2.6-2.8% w/w of histidine buffer keeping the pH at about 5.5, about 16.5 and 17.5% w/w of sucrose, about 13.3-13.5% w/w of glycine, and 0.3-0.4% w/w of polysorbate 20.

The skilled person will understand that the total combined amount of components in any given composition in % w/w of the formulation components will be equal to 100%. It should be understood also that the expression “w/w of a buffer keeping the pH between . . . ” in the context of a freeze-dried formulation according to the current invention, refers to the pH of the starting liquid formulation from which the freeze-dried formulation was obtained. For example, in a freeze-dried formulation that comprises about 67% w/w of a Fab-PEG or Fab′-PEG molecule, about 2.6-2.8% w/w of histidine buffer keeping the pH at about 5.5, about 16.5 and 17.5% w/w of sucrose, about 13.3-13.5% w/w of glycine, and 0.3-0.4% w/w of polysorbate 20, the histidine buffer keeping the pH at about 5.5 could comprise between about 0.3-0.4% w/w of histidine and 2.3-2.4% w/w of histidine-HCl.

In some embodiments, a freeze-dried formulation is obtained by freeze-drying a liquid pharmaceutical composition according to the present invention as a whole. Therefore, also encompassed is a freeze-dried formulation obtained by freeze-drying a liquid pharmaceutical composition comprising:
a. a Fab-PEG or Fab′-PEG molecule at a concentration of about 50 to about 200 mg/mL,
b. a buffer keeping the pH between about 5.0 to about 7.0,
c. from about 1 to about 5% w/v of sucrose,
d. from about 0.5 to about 4% w/v of glycine, and
e. optionally a surfactant.

In some embodiments, a freeze-dried formulation is obtained by freeze-drying a liquid pharmaceutical composition comprising or consisting of a Fab-PEG or Fab′-PEG molecule at about 50 to about 200 mg/mL, about 10 to about 100 mM of a buffer (such as an histidine buffer) at pH of about 5.0 to about 7.0, about 1 to about 5% w/v of sucrose, about 0.5 to about 4% w/v of glycine and optionally about 0.01 to about 0.2% w/v of PS20. In some embodiments, a freeze-dried formulation is obtained by freeze-drying a liquid pharmaceutical composition comprising or consisting of a Fab-PEG or Fab′-PEG molecule at about 70 to about 150 mg/mL, about 10 to about 50 mM of a buffer (such as an histidine buffer) at pH of about 5.0 to about 6.0, about 1.5 to about 4% w/v of sucrose, about 1 to about 3% w/v of glycine and optionally about 0.02 to about 0.1% w/v of PS20. In some embodiments, a freeze-dried formulation is obtained by freeze-drying a liquid pharmaceutical composition comprising or consisting of a Fab-PEG or Fab′-PEG molecule at about 70 to about 150 mg/mL, about 10 to about 50 mM of a buffer (such as an histidine buffer) at pH of about 5.0 to about 6.0, about 2 to about 4% w/v of sucrose, about 1.5 to about 3% w/v of glycine and optionally about 0.02 to about 0.1% w/v of PS20. For example, a freeze-dried formulation is obtained by freeze-drying a liquid pharmaceutical composition comprising about 100 mg/mL of the Fab-PEG or Fab′-PEG molecule, about 20 mM of Histidine buffer which keep the pH at about 5.5, about 2.5% w/v of sucrose, about 2% w/v of glycine and about 0.05% w/v of polysorbate 20. In some embodiments, a freeze-dried formulation is obtained by freeze-drying a liquid pharmaceutical composition comprising about 100 mg/ml of the Fab-PEG or Fab′-PEG molecule, about 20 mM of Histidine buffer which keep the pH at about 5.5, about 2.5% w/v of sucrose and about 2% w/v of glycine.

The invention further provides methods for manufacturing any of the above described pharmaceutical compositions. In some embodiments, the methods comprise a step of forming a mixture of Fab-PEG or Fab′-PEG molecule together with a buffer, sucrose, glycine and a surfactant. The manufacturing step is typically carried out using conventional procedures. In some embodiments, in order to prepare a suitable liquid pharmaceutical composition according to the invention, a given amount of a Fab-PEG or Fab′-PEG molecule, such as about 50 to about 200 mg/mL, is mixed with about 10 to about 100 mM of a buffer (such as an histidine buffer) which keeps the pH at about 5.0 to about 7.0, about 1 to about 5% w/v of sucrose, about 0.5 to about 4% w/v of glycine and optionally a surfactant. Each one of these compounds (i.e. Fab-PEG or Fab′-PEG molecule, buffer, sucrose, glycine and optional surfactant) can be used according to the concentrations, pH, and/or ratios above described. The resulting mixture is then dispensed into vials. Variations of this process will be recognized by one of ordinary skill in the art.

The invention further provides methods for manufacturing any of the above described lyophilised formulations (i.e. lyophilizates). In some embodiments, such methods comprise steps of: 1) forming a mixture of a Fab-PEG or Fab′-PEG molecule together with a buffer, sucrose, glycine and an optional surfactant, 2) submitting the mixture of step 1) to freeze-drying, and 3) recovering the freeze-dried formulation. The manufacturing steps are carried out using conventional procedures. In some embodiments, in order to prepare a suitable stable formulation, 1) a given amount of a Fab-PEG or Fab′-PEG molecule, such as about 50 to about 200 mg/mL, is mixed with about 10 to about 100 mM of a buffer (such as an histidine buffer) which keeps the pH at about 5.0 to about 7.0, about 1 to about 5% w/v of sucrose, about 0.5 to about 4% w/v of glycine and optionally about 0.01 to about 0.2% w/v of PS20, 2) the resulting mixture of step 1) is submitted to lyophilisation and 3) the lyophilizate is recovered. Each one of the compounds (i.e. the Fab-PEG or Fab′-PEG molecule, buffer, sucrose, glycine and optional surfactant) can be used according to the concentrations, pH, and/or ratios above described. The resulting lyophilizate is then dispensed into vials. Variations of this process will be recognized by one of ordinary skills in the art.

Additional excipients for use within the liquid pharmaceutical compositions or freeze-dried formulations according to the invention include, but are not limited to, viscosity enhancing agents, bulking agents, solubilising agents and/or combinations thereof.

The present invention also provides for containers comprising pharmaceutical compositions according to the invention. In some embodiments, a container may be, without any limitations, a vial, an ampoule, a tube, a bottle and/or a syringe (such as a pre-filled syringe) comprising a pharmaceutical composition.

In some embodiments, a container may be part of a kit-of-parts comprising one or more containers comprising liquid pharmaceutical compositions or freeze-dried formulations according to the invention and delivery devices such as a syringe, pre-filled syringe, an autoinjector, a needleless device, an implant or a patch, and/or other devices for parental administration and instructions of use.

The invention also provides a kit of parts comprising the above described liquid pharmaceutical or freeze-dried formulations and instructions for use. In a further embodiment the kit of parts comprises the liquid or freeze-dried formulations according to the invention, in one or more containers and instructions for use.

The invention also provides an article of manufacture, for pharmaceutical or veterinary use, comprising a container comprising any of the above described liquid pharmaceutical compositions or freeze-dried formulations. Also described, a packaging material providing instructions for use.

Lyophilised formulations provided herein, in particular for single use, are suitable for pharmaceutical or veterinary use, once reconstituted with a solvent. The volume of solvent used for reconstitution dictates the concentration of the Fab-PEG or Fab′-PEG molecule in the resulting liquid pharmaceutical composition. Reconstitution with a smaller volume of solvent than the pre-lyophilization volume provides a formulation which is more concentrated than before lyophilization and vice-versa. The present invention therefore provides for methods for reconstituting a freeze-dried pharmaceutical composition according to the invention by adding a solvent. In some embodiments, the solvent is water (such as water for injection) or saline buffer (such as PBS). The reconstitution ratio (volume of pre-lyophilized pharmaceutical composition to solvent used to reconstitute the freeze-dried pharmaceutical composition) may vary from about 2:1 to about 1:10 such as for instance about 2:1, about 3:2, about 1:1 or about 1:2. Therefore, as a non-limiting example, should the pre-lyophilized pharmaceutical composition comprise or consist of 100 mg/ml of the Fab-PEG or Fab′-PEG molecule, 20 mM of Histidine buffer which keep the pH at about 5.5, 2.5% w/v of sucrose, 2% w/v of glycine and 0.05% w/v of polysorbate 20, and should the reconstitution ratio be 1:1, the reconstituted formulation would contain 100 mg/mL of the Fab-PEG or Fab′-PEG molecule, 20 mM of Histidine buffer which keep the pH at about 5.5, 2.5% w/v of sucrose, 2% w/v of glycine and 0.05% w/v of polysorbate 20.

The present invention further describes a freeze-dried formulation, characterized in that after reconstitution of said freeze-dried formulation, the resulting liquid formulation comprises a Fab-PEG or Fab′-PEG molecule at about 70 to about 150 mg/mL, about 10 to about 50 mM of a buffer (such as an histidine buffer) at pH of about 5.0 to about 6.0, about 2 to about 4% w/v of sucrose, about 1.5 to about 3% w/v of glycine and optionally about 0.02 to about 0.1% w/v of surfactant (such as PS20). For instance, a freeze-dried pharmaceutical composition after reconstitution according to the invention can comprise about 100 mg/ml of a Fab-PEG or Fab′-PEG molecule, about 20 mM of Histidine buffer which keeps the pH at about 5.5, about 2.5% w/v of sucrose, about 2% w/v of glycine and about 0.05% w/v of polysorbate 20. In some embodiments, a freeze-dried pharmaceutical composition after reconstitution according to the invention can comprise about 100 mg/ml of a Fab-PEG or Fab′-PEG molecule, about 20 mM of Histidine buffer which keeps the pH at about 5.5, about 2.5% w/v of sucrose and about 2% w/v of glycine. In some embodiments, a freeze-dried pharmaceutical composition after reconstitution comprises or consists of about 50 to about 200 mg/ml of a Fab-PEG or Fab′-PEG molecule, about 10 to about 100 mM of a buffer (such as an histidine buffer) at pH of about 5.0 or about 7.0, sucrose and glycine in a respective ratio (w/w) of about 2:3 to about 2:1 and optionally about 0.01 to about 0.2% w/v of surfactant (such as PS20). In some embodiments, a freeze-dried pharmaceutical composition after reconstitution comprises or consists of about 70 to about 150 mg/ml of a Fab-PEG or Fab′-PEG molecule, about 10 to about 50 mM of a buffer (such as an histidine buffer) at pH of about 5.0 to about 6.0, sucrose and glycine in a respective ratio (w/w) of about 3:4 to about 5:3 and optionally about 0.02 to about 0.1% w/v of surfactant (such as PS20). In some embodiments, the freeze-dried pharmaceutical composition after reconstitution of the present invention comprises or consists of a Fab-PEG or Fab′-PEG molecule at about 70 to about 150 mg/mL, about 10 to about 50 mM of a buffer (such as an histidine buffer) at pH of about 5.0 to about 6.0, sucrose and glycine in a respective ratio (w/w) of about 4:5 to about 3:2 and optionally about 0.02 to about 0.1% w/v of surfactant (such as PS20). For instance, a freeze-dried pharmaceutical composition after reconstitution according to the invention can comprise about 100 mg/mL of a Fab-PEG or Fab′-PEG molecule, about 20 mM of Histidine buffer which keep the pH at about 5.5, sucrose and glycine in a respective ratio (w/w) of 1:1 or about 5:4 and about 0.05% w/v of polysorbate 20. In some embodiments, a freeze-dried pharmaceutical composition after reconstitution according to the invention can comprise about 100 mg/ml of a Fab-PEG or Fab′-PEG molecule, about 20 mM of Histidine buffer which keeps the pH at about 5.5 and sucrose and glycine in a respective ratio (w/w) of about 1:1 or about 5:4. In other embodiments, a freeze-dried pharmaceutical composition after reconstitution according to the invention can comprise about 70 mg/ml of Fab-PEG or a Fab′-PEG molecule, about 14 mM of Histidine/Histidine HCl buffer which keeps the pH at about 5.5 and sucrose and glycine in a respective ratio (w/w) of about 1:1 or about 5:4.

In some embodiments, lyophilised formulations of the invention may be kept for at least about 12 months to about 24 months. In some embodiments, under preferred storage conditions, before the first use, the formulations are kept away from bright light (preferably in the dark), at temperature from about 2 to about 25° C., e.g. at room temperature (about 20-25° C.) or at about 2-8° C. (see following examples).

Pharmaceutical compositions and/or reconstituted lyophilizates according to the invention are for use in therapy. For example, the pharmaceutical composition or the reconstituted lyophilizates according to the invention can be used in the treatment of various disorders or diseases such as an autoimmune disease, an inflammatory disease, a cancer, a neoplastic disease and/or a neurodegenerative disease. The invention also provides methods for treating various disorders or diseases such as an autoimmune disease, an inflammatory disease, a cancer, a neoplastic disease and/or a neurodegenerative disease in a mammalian subject comprising administering a pharmaceutical composition and/or reconstituted lyophilizate according to the invention. In some embodiments, pharmaceutical compositions and/or reconstituted lyophilizates according to the invention may be used in the manufacture of a medicament for the treatment of various disorders or diseases such as an autoimmune disease, an inflammatory disease, a cancer, a neoplastic disease and/or a neurodegenerative disease.

In the context of the invention as a whole, autoimmune diseases or inflammatory diseases include but are not limited lupus, including systemic lupus erythematosus (SLE), cutaneous lupus (CLE), drug-induced lupus erythematosus, or neonatal lupus, rheumatoid arthritis, ankylosing spondylitis, lupus nephritis, Sjögren's syndrome, polymyositis, dermatomyositis, temporal arteritis, ANCA-associated vasculitis, Churg-Strauss syndrome, antiphospholipid syndrome, membranous glomerulonephropathy, Goodpasture's disease, immunoglobulin A nephropathy, Henoch-Schönlein purpura, chronic graft rejection, atopic dermatitis, pemphigus vulgaris, psoriasis, asthma, allergy, systemic sclerosis, multiple sclerosis, Guillain-Barré syndrome, transverse myelitis, chronic immune polyneuropathy, myasthenia gravis, Addison's disease, thyroiditis, autoimmune gastritis, pernicious anaemia, celiac disease, ulcerative colitis, sarcoidosis, hemolytic anemia, idiopathic thrombocytopenia purpura, Behçet's disease, primary biliary cirrhosis, autoimmune diabetes, Lyme neuroborreliosis, interstitial lung disease. Neurodegenerative diseases include but are not limited to Alzheimer's disease, Parkinson's disease, Friedreich's ataxia, Huntington's disease, amyotrophic lateral sclerosis (ALS), myasthenia gravis, multifocal motor neuropathy, primary lateral sclerosis, multiple sclerosis, spinal muscular atrophy, Kennedy's disease, and spinocerebellar ataxia. Similarly, conditions such as atherosclerosis, heart failure, osteoarthritis, nonalcoholic steatohepatitis, irritable bowel syndrome, Crohn's disease, diabetic complications (nephropathy, neuropathy, arteriopathy, retinopathy), asthma, cystic fibrosis, chronic obstructive airway disease, epilepsy, glaucoma, age-related macular degeneration, psychiatric disorders (anxiety, depression, psychosis), chronic fatigue syndrome, enthesiopathies/tendinopathies, prematurity/prenatal infection, obesity/metabolic syndrome, dermatological conditions (acne vulgaris, acne rosacea, solar keratosis), abnormal wound healing (keloid scarring), urogenital disorders (prostatism/prostatitis, overactive bladder syndrome).

In the context of the invention as a whole, pharmaceutical compositions and/or reconstituted freeze-dried formulations (lyophilizates) according to the invention are for use in a method of treating an autoimmune inflammatory, neurodegenerative or neuromuscular disorder in a mammalian subject, which method comprises administering a dose of about 24 mg/kg of Fab-PEG or Fab′-PEG that specifically binds to CD40L to a subject in need of such treatment, with a frequency of about once every 4 weeks. In some embodiments a freeze-dried formulation according to the invention is reconstituted according to the instructions for use, and added to a saline solution bag for intravenous administration.

In certain embodiments, freeze-dried formulations of the present invention comprising about 67% w/w of Fab-PEG or Fab′-PEG that specifically binds to CD40L, about 2.6-2.8% w/w of histidine/Histidine HCl buffer keeping the pH at about 5.5, about 16.5 and 17.5% w/w of sucrose, about 13.3-13.5% w/w of glycine, and 0.3-0.4% w/w of polysorbate 20 are reconstituted according to the instructions for use, for use in a method of treating an autoimmune disease such as systemic lupus erythematosus in a mammalian subject, said method comprising administering a dose of 24 mg/kg of Fab-PEG or Fab′-PEG that specifically binds to CD40L to a subject in need of such treatment, with a frequency of about once every 4 weeks. In a certain embodiments the reconstituted freeze-dried formulation of the present invention is administered for at least 24 weeks, or for at least 48 weeks.

In a particular embodiment, the Fab-PEG or Fab′-PEG that specifically binds to CD40L:
a) comprises CDR-H1 having the sequence as defined in SEQ ID NO: 1; CDR-H2 having the sequence as defined in SEQ ID NO: 2; CDR-H3 having the sequence as defined in SEQ ID NO: 3; CDR-L1 having the sequence as defined in SEQ ID NO: 4; CDR-L2 having the sequence as defined in SEQ ID NO: 5 and CDR-L3 having the sequence as defined in SEQ ID NO: 6; or
b) comprises a light variable region having the sequence as defined in SEQ ID NO: 7 and a heavy variable region having the sequence as defined in SEQ ID NO: 8; or
c) comprises a light variable region having at least 80% identity, preferably 90% identity to the sequence as defined in SEQ ID NO: 7 and a heavy variable region having at least 80% identity, preferably 90% identity to the sequence as defined in SEQ ID NO: 8; or
d) comprises a light chain as defined in SEQ ID NO: 9 and a heavy chain as defined in SEQ ID NO: 10.

In some embodiments the Fab-PEG or Fab′-PEG molecule comprises
a) a maleimide group covalently linked to a single thiol group in the modified hinge region; a lysine residue is covalently linked to the maleimide group; and
b) a methoxypoly(ethyleneglycol) polymer having a molecular weight of approximately 20 KDa is attached to each of the amine groups on the lysine residue.

In a further embodiment, the reconstituted freeze-dried formulations of the present invention are for use in a method of treatment of an autoimmune inflammatory, neurodegenerative or neuromuscular disorder in combination additional therapeutic agents. For example, the reconstituted freeze-dried formulations of the present invention may be co-administered with a corticosteroid, a non-steroidal anti-inflammatory drug (NSAID) or therapies targeting the many proinflammatory cytokines known to be involved in the pathogenesis of immune-mediated disorders, such as TNF, IL-1, IL-6, CTLA-4, JAK, IFN (e.g. anifrolumab) or those targeting B-cell activity such as CD19, CD20 (e.g. rituximab), CD22, BAFF (e.g. belimumab) and BlyS/APRIL (e.g. atacicept).

In some embodiments, freeze-dried formulations of the present invention have improved stability and can be easily stored at a temperature from about 2 to about 25° C., such as at room temperature (about 20-25° C.) or at about 2-8° C. (see following examples). Indeed, the inventors have found that lyophilised formulations comprising about 67% w/w of a Fab-PEG or Fab′-PEG molecule, about 2.5-2.6% w/w of a buffer keeping the pH between about 5.0 to about 7.0, about 16.5-16.75% w/w of sucrose, about 13.3-13.5% w/w of glycine, and optionally about 0.3% w/w of a surfactant are stable over time, notably when stored at a temperature from about 2 to about 25° C. Said formulations minimize the aggregation of the Fab-PEG or Fab′-Peg molecule but also reduce the time for reconstitution.

The following examples are provided to further illustrate the preparation of the formulations and compositions of the invention. The scope of the invention shall not be construed as merely consisting of the following examples.

### Description of the Sequences:

SEQ ID NO: 1: CDR-H1
GFSSTNYHVH
SEQ ID NO: 2: CDR-H2
VIWGDGDTSYNSVLKS
SEQ ID NO: 3: CDR-H3
QLTHYYVLAA
SEQ ID NO: 4: CDR-L1
RASEDLYYNLA
SEQ ID NO: 5: CDR-L2
DTYRLAD
SEQ ID NO: 6: CDR-L3
QQYYKFPFT
SEQ ID NO: 7: variable light chain
DIQMTQSPSSLSASVGDRVTITCRASEDLYYNLAWYQRKPGKAPKLLIY
DTYRLADGVPSRFSGSGSGTDYTLTISSLQPEDFASYYCQQYYKFPFTF
GQGTKVEIK
SEQ ID NO: 8: variable heavy chain
EVQLVESGGGLVQPGGSLRLSCAVSGFSSTNYHVHWVRQAPGKGLEWMG
VIWGDGDTSYNSVLKSRFTISRDTSKNTVYLQMNSLRAEDTAVYYCARQ
LTHYYVLAAWGQGTLVTVSS
SEQ ID NO: 9: Fab-light chain
DIQMTQSPSSLSASVGDRVTITCRASEDLYYNLAWYQRKPGKAPKLLIY
DTYRLADGVPSRFSGSGSGTDYTLTISSLQPEDFASYYCQQYYKFPFTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
SEQ ID NO: 10: Fab'-heavy chain
EVQLVESGGGLVQPGGSLRLSCAVSGFSSTNYHVHWVRQAPGKGLEWMG
VIWGDGDTSYNSVLKSRFTISRDTSKNTVYLQMNSLRAEDTAVYYCARQ
LTHYYVLAAWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCAA

### Examples

### Abbreviations

DLS (Dynamic Light Scattering); SEC (Size-exclusion Chromatography); SE-HPLC (Size-exclusion high-performance liquid chromatography); CEX: cation exchange chromatography; Suc (Sucrose); PS20 (Polysorbate 20); Gly (Glycine); Man (Mannitol); Tre (Trehalose); His (Histidine); Lac (Lactose); Cit (citrate); Ace (acetate); Pov (povidone); Aggr: Aggregation. FD: freeze-drying or freeze-dried; conc: concentration; prelyo: pre-lyophilisation; recon: reconstitution.

### Material

The active pharmaceutical ingredient is a Fab′-PEG, herein named Fab1. Its light chain sequence is shown in SEQ ID NO: 9 and its heavy chain sequence is shown in SEQ ID NO: 10. This molecule is PEGylated at a cysteine in the modified hinge region as described in WO 2008/118356. A maleimide group is covalently linked to a single thiol group in a cysteine in the modified hinge region; a lysine residue is covalently linked to the maleimide group; and a methoxypoly(ethyleneglycol) polymer having a molecular weight of approximately 20 KDa is attached to each of the amine groups on the lysine residue. The total molecular weight of the entire PEG covalently linked to the monovalent Fab′ is therefore approximately 40 KDa. It is presented, before formulation work, as a liquid composition in 50 mM sodium acetate, 125 mM sodium chloride, pH 5.0 (herein named DS).

### Methods

### Reconstitution:

The lyophilized product was reconstituted using a standard method, i.e. by injecting water into the vial. The time for the cake to completely dissolve to a clear solution was measured with a stopwatch.

### Appearance (Pre- and Post-Reconstitution):

Structural integrity of cake was checked visually, and any defects reported. Observations were supported by photographic material. Appearance analysis of reconstituted samples was performed to confirm colour and detect visible particles. The appearance analysis was based on a visual assessment.

### Protein Concentration:

Protein concentration was determined by measuring the absorption at 280 nm after dilution to approximately 1 mg/mL and analysed using a spectrophotometer that has been calibrated, according to standard methods. In the case where the sample is visibly aggregated, samples may need to be centrifuged briefly to remove the precipitate and the concentration determined on the aspirated supernatant. The sample concentration is then calculated with the following formula using the established extinction coefficient:

$$c = \frac{{A}_{280} xdil}{{E}_{m} xb}$$

(with c=protein concentration (mg/mL); A₂₈₀=absorbance at 280 nm; dil=dilution factor; Eₘ=extinction coefficient (0.82 mL/mg·cm); b=path length (cm)).

### Protein Aggregation, High Molecular Weight Species and Low Molecular Weight Species:

SEC or SE-HPLC were used according to standard methods, to resolve and quantitate product-related species (e.g., low molecular weight species, aggregates and high molecular weight species) in reconstituted formulations. Detection and quantitation were performed by UV absorbance at 214 nm.

### Acidic and Basic Species:

CEX analysis was used to determine the charge profile and provide values for total acidic and basic species, according to standard methods (using fluorescence detection at emission at 340 nm and excitation at 280 nm).

### Example 1: Preliminary Screening

Formulation development was started with a preliminary screening study to preselect at least the buffer and the stabilizer (See Table 1). Standard procedures were applied for the lyophilisation. Prelyophilisation formulations contained 100 mg/mL of Fab1 and were prepared by buffer exchange.

TABLE 1
Preliminary screening formulations

| | Buffer | Stabilizer | Bulking agent | Surfactant |
|---|---|---|---|---|
| 1 | 20 mM His pH6.0 | 60 mM Suc | 2% Man | 0.01% Tween |
| 2 | 20 mM His pH6.0 | 60 mM Suc | None | 0.01% Tween |
| 3 | 20 mM His pH6.0 | 60 mM Suc | 2% Pov | 0.01% Tween |
| 4 | 20 mM His pH6.0 | 60 mM Tre | 2% Man | None |
| 5 | 20 mM His pH6.0 | 60 mM Tre | 2% Man | None |
| 6 | 20 mM His pH6.0 | 60 mM Tre | None | None |
| 7 | 20 mM His pH6.0 | 60 mM Tre | None | None |
| 8 | 20 mM His pH6.0 | 60 mM Tre | 2% Pov | None |
| 9 | 20 mM His pH6.0 | 60 mM Tre | 2% Pov | None |
| 10 | 20 mM Lac pH4.5 | 60 mM Suc | 2% Man | None |
| 11 | 20 mM Lac pH4.5 | 60 mM Suc | None | None |
| 12 | 20 mM Lac pH4.5 | 60 mM Suc | 2% Pov | None |
| 13 | 20 mM Lac pH4.5 | 60 mM Tre | 2% Man | 0.01% Tween |
| 14 | 20 mM Lac pH4.5 | 60 mM Tre | 2% Man | 0.01% Tween |
| 15 | 20 mM Lac pH4.5 | 60 mM Tre | None | 0.01% Tween |
| 16 | 20 mM Lac pH4.5 | 60 mM Tre | None | 0.01% Tween |
| 17 | 20 mM Lac pH4.5 | 60 mM Tre | 2% Pov | 0.01% Tween |
| 18 | 20 mM Lac pH4.5 | 60 mM Tre | 2% Pov | 0.01% Tween |

Lyophilized samples were reconstituted by injecting 0.5 ml water into the vial for the formulation having a concentration of Fab1 at 100 mg/mL and by injecting 1.0 ml water into the vial for the formulation having a concentration of antibody at 50 mg/mL.

The lyophilization formulations were evaluated on the cake appearance of the lyophilized product. They appeared as an acceptable, white in colour, well-formed cake without defects (pictures not shown).

TABLE 2
Reconstitution time and Fab1 concentration after reconstitution

| | Target concentration: 50 mg/mL | | Target concentration: 100 mg/mL | |
|---|---|---|---|---|
| | | Fab1 | | Fab1 |
| | Reconstitution | concentration | Reconstitution | concentration |
| | time (min) | (mg/mL)^{a} | time (min) | (mg/mL)^{a} |
| DS | 7.1 | 48.7 | 20.6 | 100.1 |
| 1 | 1.8 | 45.1 | 2.9 | 87.4 |
| 2 | 1.7 | 47.8 | 4.5 | 90.7 |
| 3 | 1.3 | 44.7 | 9.6 | 84.3 |
| 4 | 1.4 | 48.7 | 5.1 | 93.2 |
| 5 | 1.6 | 49.8 | 5.8 | 91.7 |
| 6 | 1.3 | 48.8 | 3.8 | 90.5 |
| 7 | 1.5 | 44.4 | 5.6 | 87.8 |
| 8 | 2.4 | 46.3 | 9.7 | 93.2 |
| 9 | 1.6 | 45.9 | 12.4 | 91.3 |
| 10 | 0.7 | 44.5 | 3.4 | 88.9 |
| 11 | 1.0 | 46.2 | 7.7 | 93.5 |
| 12 | 1.2 | 44.9 | 11.4 | 94.9 |
| 13 | 0.8 | 41.8 | 4.3 | 87.2 |
| 14 | 0.8 | 43.0 | 3.6 | 90.9 |
| 15 | 0.9 | 47.2 | 4.7 | 88.0 |
| 16 | 1.2 | 46.0 | 6.9 | 100.1 |
| 17 | 1.6 | 45.5 | 11.3 | 90.9 |
| 18 | 1.7 | 42.7 | 11.6 | 84.3 |

^{a}Protein concentrations were typically lower than in the initial lyophilization formulation due to volume effect of lyophilized cake.

The samples were then evaluated for reconstitution time and protein concentration (see Table 2).

As it can be observed, there is a dramatic increase in reconstitution time between 50 mg/mL formulations and 100 mg/mL formulations, reconstitution time being more pronounced for the 100 mg/mL formulations. It is noted that when reconstituted with 0.5 ml water, the solutions were diluted with respect to the prelyophilisation formulation.

Protein aggregation was also determined for the formulations with 100 mg/ml of antibody (see Table 3). No significant difference in protein aggregation was observed for the different formulations.

TABLE 3
Protein aggregation before lyophilization/after reconstitution

| | Aggregation (%; SE-HPLC) | |
|---|---|---|
| | Before lyophilisation | After reconstitution |
| DS | 0.5 | N/A |
| 1 | 0.6 | 0.6 |
| 2 | 0.6 | 0.7 |
| 3 | —^{a} | —^{a} |
| 4 | 0.6 | 0.7 |
| 5 | 0.6 | 0.7 |
| 6 | 0.6 | 0.7 |
| 7 | 0.6 | 0.7 |
| 8 | —^{a} | —^{a} |
| 9 | —^{a} | —^{a} |
| 10 | 0.5 | 0.6 |
| 11 | 0.5 | 0.6 |
| 12 | —^{a} | —^{a} |
| 13 | 0.5 | 0.5 |
| 14 | 0.5 | 0.6 |
| 15 | 0.5 | 0.6 |
| 16 | 0.5 | 0.6 |
| 17 | —^{a} | —^{a} |
| 18 | —^{a} | —^{a} |

^{a}No data, interference by Povidone in chromatogram

Since no significant differences were seen in protein aggregation only the reconstitution time was considered to compare the different formulations. As was already clear from a first examination of the data povidone had a negative effect on the reconstitution time. Mannitol showed to have a positive effect on the reconstitution time. The model gave a preference for histidine as buffer and sucrose as stabilizer. The effect of surfactant (0.01% Tween 20) was not significant.

### Example 2: Optimisation Screening

The screening study (Example 1) indicated the optimal preformulation to be a sucrose/mannitol formulation and was further evaluated for optimization. Since high pH formulations posed a problem for the stability of the product with respect to an increase in acidic species, other buffers besides histidine pH6.0, were also taken into account for the formulation optimization and evaluated for an increase in acidic species (see formulations in Table 4).

TABLE 4
Optimization formulations

| | | | | Bulking | | Protein |
|---|---|---|---|---|---|---|
| | Buffer | pH | Stabilizer | agent | Surfactant | concentration |
| 20 | 20 mM Lac | 4.5 | 1% Suc | 4% Man | N/A | 100 mg/mL |
| 21 | 20 mM Lac | 4.5 | 2.5% Suc | 2.5% Man | N/A | 100 mg/mL |
| 22 | 20 mM Lac | 4.5 | 4% Suc | 1% Man | N/A | 100 mg/mL |
| 23 | 20 mM Cit | 5.25 | 1% Suc | 4% Man | N/A | 100 mg/mL |
| 24 | 20 mM Cit | 5.25 | 2.5% Suc | 2.5% Man | N/A | 100 mg/mL |
| 25 | 20 mM Cit | 5.25 | 2.5% Suc | 2.5% Man | N/A | 100 mg/mL |
| 26 | 20 mM Cit | 5.25 | 4% Suc | 1% Man | N/A | 100 mg/mL |
| 27 | 20 mM His | 6.0 | 1% Suc | 4% Man | N/A | 100 mg/mL |
| 28 | 20 mM His | 6.0 | 2.5% Suc | 2.5% Man | N/A | 100 mg/mL |
| 29 | 20 mM His | 6.0 | 4% Suc | 1% Man | N/A | 100 mg/mL |
| 30 | 20 mM Cit | 5.25 | 5% Suc | — | N/A | 100 mg/mL |
| 31 | 20 mM Cit | 5.25 | None | 5% Man | N/A | 100 mg/mL |
| 32 | 20 mM Cit | 5.25 | 2.5% Suc | 2.5% Man | 0.01% PS20 | 100 mg/mL |
| 33 | 20 mM Cit | 5.25 | 2.5% Suc | 2.5% Man | 0.05% PS20 | 100 mg/mL |
| 34 | 20 mM Cit | 5.25 | 2.5% Suc | 2.5% Man | N/A | 150 mg/mL |
| 35 | 20 mM Cit | 5.25 | 2.5% Suc | 2.5% Man | N/A | 200 mg/mL |

All samples were evaluated on reconstitution time, protein aggregation and acidic species (see Table 5; value at t0).

TABLE 5
Reconstitution time (Recon time), protein concentration
after reconstitution (Fab1 conc) and aggregation
and acidic species before lyophilization (prelyo)/after
reconstitution (after recon)

| | | Fab1 conc | Aggregation | | Acidic species^{b} | |
|---|---|---|---|---|---|---|
| | Recon | after | (%; SEC) | | (%, CEX) | |
| | time^{a} | recon | | After | | After |
| | (min) | (mg/ml) | prelyo | recon | prelyo | recon |
| DS | 24.9 ± 5.2 | 98.9 | 0.5 | N/A | 10.2 | N/A |
| 20 | 6.7 ± 0.3 | 88.3 | 0.4 | 1.0 | 10.0 | 10.7 |
| 21 | 6.5 ± 0.9 | 90.1 | 0.4 | 0.6 | 10.2 | 10.5 |
| 22 | 7.7 ± 1.3 | 94.9 | 0.4 | 0.5 | 10.2 | 10.3 |
| 23 | 4.9 ± 2.0 | 70.0 | 0.5 | 0.8 | 10.5 | 10.9 |
| 24 | 5.6 ± 0.9 | 78.5 | 0.5 | 0.5 | 10.6 | 11.0 |
| 25 | 7.9 ± 0.4 | 91.0 | 0.5 | 0.5 | 10.4 | 10.9 |
| 26 | 10.3 ± 1.9 | 84.6 | 0.5 | 0.5 | 10.8 | 11.1 |
| 27 | 5.3 ± 0.5 | 91.8 | 0.6 | 0.9 | 13.7 | 13.6 |
| 28 | 4.1 ± 0.5 | 92.6 | 0.6 | 0.7 | 13.3 | 13.4 |
| 29 | 5.8 ± 2.1 | 88.7 | 0.6 | 0.6 | 13.6 | 12.7 |
| 30 | 14.1 ± 4.7 | 88.7 | 0.5 | 0.5 | 10.8 | 11.1 |
| 31 | 5.0 ± 2.1 | 94.3 | 0.5 | 1.8 | 10.4 | 11.3 |
| 32 | 10.6 ± 2.9 | 94.9 | 0.5 | 0.6 | 11.6 | 10.4 |
| 33 | 7.4 ± 0.2 | 84.9 | 0.5 | 0.5 | 10.2 | 10.6 |
| 34 | 54 ± 24 | 116.3 | 0.5 | 0.7 | 8.4 | 10.8 |
| 35 | >60 | 175.6 | 0.5 | 0.8 | 10.7 | 11.1 |

^{a}Reconstitution time is the mean of three measurements
^{b}CEX analysis was delayed which resulted in a higher acidic species content, especially for formulations 8-10.

All the formulations were then evaluated on their capacity to stabilize and protect the Fab-PEG product against degradation with respect to protein aggregation and increases in acidic species over time (Table 6). Liquid samples (formulations not lyophilised) and lyophilized samples were incubated at 2-8° C. and 30° C. during four weeks.

TABLE 6
Accelerated stability: protein aggregation and acidic species
(after 4 weeks, T0 values are reported in Table 5).

| | Aggregation (%, SE-HPLC) | | | | Acidic species (%, CEX) | | | |
|---|---|---|---|---|---|---|---|---|
| | Liquid | | Lyophilized | | Liquid | | Lyophilized | |
| | 2-8° C.^{a} | 30° C.^{a} | 2-8° C.^{a} | 30° C.^{a} | 2-8° C.^{b} | 30° C.^{b} | 2-8° C. | 30° C. |
| DS | 0.4 | 1.2 | 1.8 | 9.7 | 10.5 | 21.4 | — | — |
| 20 | 0.3 | 0.4 | 0.3 | 0.7 | 8.4 | 16.8 | 10.9 | 17.7 |
| 21 | 0.2 | 0.3 | 0.3 | 0.5 | 10.2 | 17.3 | 10.0 | 14.0 |
| 22 | 0.2 | 0.3 | 0.3 | 0.4 | 10.2 | 16.4 | 9.4 | 12.5 |
| 23 | 0.3 | 0.6 | 0.4 | 0.7 | 9.4 | 30.3 | 9.6 | 14.4 |
| 24 | 0.2 | 0.4 | 0.3 | 0.9 | 10.8 | 30.7 | 10.2 | 13.7 |
| 25 | 0.2 | 0.4 | 0.3 | 0.4 | 10.5 | 30.7 | 8.8 | 12.9 |
| 26 | 0.2 | 0.4 | 0.2 | 0.7 | 9.5 | 30.8 | 8.8 | 13.3 |
| 27 | 0.2 | 0.4 | 0.4 | 0.4 | 15.8 | 52.1 | 9.9 | 14.7 |
| 28 | 0.2 | 0.3 | 0.3 | 0.3 | 16.2 | 52.0 | 11.0 | 11.8 |
| 29 | 0.2 | 0.3 | 0.2 | 0.2 | 16.0 | 50.6 | 11.0 | 8.3 |
| 30 | 0.2 | 0.4 | 0.2 | 0.4 | 10.7 | 29.2 | 10.4 | 11.7 |
| 31 | 0.2 | 0.4 | 0.9 | 2.5 | 10.7 | 29.2 | 9.9 | 17.6 |
| 32 | 0.2 | 0.4 | N/A | 0.7 | 11.2 | 30.0 | N/A | 14.8 |
| 33 | 0.2 | 0.4 | N/A | 0.8 | 11.7 | 31.2 | N/A | 12.5 |
| 34 | 0.2 | 0.5 | N/A | 0.8 | N/A | 29.0 | N/A | 14.5 |
| 35 | 0.2 | N/A | N/A | N/A | N/A | N/A | N/A | N/A |

^{a}SEC column performance was reduced which explains the apparent lower content of aggregation species after 4 weeks stability compared to t0. However the data still allow comparison between different formulations.
^{b}CEX analysis was delayed which resulted in a higher acidic species content, especially for formulations 8-10.

While for the DS formulation (100 mg/mL Fab1, 50 mM Na-Acetate, 125 mM NaCl, pH5.0) an important increase in protein aggregation and acidic species was observed after four weeks at 30° C., most of the lyophilized products showed to be stable. Formulations containing higher sucrose:mannitol ratio showed to be more stable than formulations containing a low sucrose:mannitol ratio. In particular the formulation containing only mannitol showed an important increase in protein aggregation and acidic species (Table 6, formulation 31). Formulations containing histidine buffer proved to be more stable than lactate or citrate buffer. For liquid formulations, a higher pH resulted in an accelerated increase in acidic species, in particular histidine formulations gave rise to more than 50% acidic species. This shows that removing the water by lyophilization is the most important factor in stabilizing the DP against an increase in acidic species.

As in example 1, the reconstitution time was the shortest for the formulations comprising histidine buffer (Table 5).

Considering both reconstitution time and stability aspects, histidine pH6.0 showed to be the best choice as a buffer. The ratio of sucrose:mannitol was a compromise between reconstitution time (low sucrose:mannitol ratio) and stability (high sucrose:mannitol ratio). The optimal composition of the lyophilization formulation was determined as 20 mM histidine pH 6.0, 2.5% sucrose, 2.5% mannitol, 100 mg/mL of the antibody molecule. The reconstitution time for this formulation was less than 5 min (reconstituted with 0.5 mL of water) and was stable against an increase in acidic species even after 4 weeks at 30° C.

### Example 3: Further Optimisation Studies

An important issue with the molecule is its stability with particular regard to hydrolysis and ring opening of the succinimide PEG linker which results in an increase in the acidic species levels. Hydrolysis of the linker is strongly pH dependent. At increasing pH, hydrolysis is accelerated. The rate of increase in acidic species was reduced in a freeze-dried formulation. However, to minimize hydrolysis of the linker during formulation preparation and after reconstitution it was recommended to reduce the pH of the freeze-drying formulation as well as to identify a possible further optimised formulation. In this regard, additional formulations comprising glycine instead of mannitol were assessed at different pH (Table 7).

TABLE 7
Composition of the additional formulations

| | | | Bulking | |
|---|---|---|---|---|
| Formulation | Buffer | Stabilizer | agent | Surfactant |
| A1/A2^{a} | 20 mM His pH6.0 | 2.5% suc | 1.5% Gly | 0.05% PS20 |
| B1/B2^{a} | 20 mM His pH6.0 | 2.5% suc | 2.0% Gly | 0.05% PS20 |
| C | 20 mM His pH6.0 | 2.5% suc | 2.5% Gly | 0.05% PS20 |
| D | 20 mM His pH6.0 | 2.5% suc | 2.5% Gly | None |
| E | 20 mM Lac pH4.5 | 2.5% suc | 2.5% Gly | None |
| F | 20 mM His pH6.0 | 2.0% suc | 2.0% Gly | 0.05% PS20 |
| G | 20 mM Lac pH4.5 | 2.0% suc | 2.0% Gly | 0.05% PS20 |
| H | 20 mM Lac pH4.5 | 2.5% suc | 1.5% Gly | 0.05% PS20 |

A1 and B1: freeze-drying with sublimation at −20° C.; A2 and B2: freeze-drying with sublimation at −5° C.

TABLE 8
Reconstitution time and protein concentration after reconstitution (formulations
were reconstituted to a final volume of 0.5 mL with water)

| | T₀ | | T_{4 w, 4° C.} | | T_{4 w, 40° C.} | |
|---|---|---|---|---|---|---|
| | Recon | | Recon | | Recon | |
| | time | Conc | time | Conc | time | Conc |
| Formulation | (min) | (mg/mL) | (min) | (mg/mL) | (min) | (mg/mL) |
| A1 | 4.2 | 111.0 | 7.1 | 101.5 | 4.6 | 112.5 |
| A2 | 2.4 | 103.1 | 9.7 | 94.0 | 11.2 | 95.6 |
| B1 | 2.7 | 98.2 | 4.4 | 101.0 | 2.8 | 98.4 |
| B2 | 2.6 | 100.1 | 7.9 | 95.1 | 9.1 | 91.4 |
| C | 2.6 | 106.2 | 7.9 | 97.6 | 3.6 | 100.6 |
| D | 2.5 | 95.9 | 2.8 | 95.0 | 2.6 | 100.3 |
| E | 4.1 | 110.5 | 4.9 | 109.6 | 6.2 | 110.0 |
| F | 2.5 | 99.0 | 3.7 | 92.1 | 4.7 | 95.4 |
| G | 5.0 | 103.1 | 2.8 | 94.4 | 3.9 | 96.0 |
| H | 4.6 | 97.1 | 5.2 | 101.1 | 7.0 | 103.1 |

TABLE 9
Aggregation and acidic species

| | HMWS (%; SE-HPLC) | | | Acidic species (%, CEX) | | |
|---|---|---|---|---|---|---|
| Formulation | T₀ | T_{4 w, 4° C.} | T_{4 w, 40° C.} | T₀ | Tt_{4 w, 4° C.} | T_{4 w, 40° C.} |
| DS | 0.6 | N/A | N/A | 9.9 | N/A | N/A |
| A1 | 0.5 | 0.6 | 1.1 | 9.8 | 11.1 | 14.3 |
| A2 | 0.6 | 0.6 | 1.2 | 11.4 | N/A | 14.2 |
| B1 | 0.6 | 0.6 | 1.2 | 10.7 | 11.1 | 14.5 |
| B2 | 0.6 | 0.6 | 1.2 | 11.1 | N/A | 15.1 |
| C | 0.7 | 0.6 | 1.2 | 9.9 | 11.1 | 14.5 |
| D | 0.6 | 0.6 | 1.1 | 13.1 | 10.9 | 13.9 |
| E | 0.7 | 0.6 | 1.7 | 10.3 | 10.8 | 16.8 |
| F | 0.6 | 0.6 | 1.3 | 11.1 | N/A | 16.4 |
| G | 0.5 | 0.5 | 1.8 | 10.4 | N/A | 22.3 |
| H | 0.5 | 0.5 | 1.3 | 10.5 | N/A | 18.7 |

Residual moisture was between 1 and 2% for freeze-dried formulations obtained by sublimation at −20° C. and less or equal to 1% for freeze-dried formulations by sublimation at −5° C. (data not shown).

Reconstitution times were less than 10 min or even less than 3 min for certain formulations (Table 8). After 4 weeks of storage at 2-8° C. and especially after 4 weeks at 40° C. reconstitution times were significantly increased, especially for freeze-dried formulations obtained with a lyophilization cycle with sublimation at −5° C.

For formulations containing 2.5% sucrose and 2.5% glycine osmolality was higher than 450 mOsm and pH of the reconstituted formulation was 0.2 to 0.5 higher than the targeted pH (data not shown).

Aggregation levels were slightly higher for lactate formulations, especially after 4 weeks at 40° C. (Table 9). Moreover, an important increase in levels of acidic species was observed for lactate formulations (Table 9).

Glycine didn't crystallize in formulations containing only 1.5% glycine. But in formulations with 2.0% glycine, and especially in formulations with 2.5% glycine, partial crystallization of glycine was observed (data not shown). Higher sublimation temperature didn't result in a higher content of crystalline glycine. Glycine was crystallized as the β-polymorph, which is the least stable polymorphic form of glycine.

A 100 mg/mL Fab1 in 20 mM histidine pH 6.0, 2.5% sucrose, 2.0% glycine, 0.05% Tween20 was considered a possible alternative freeze-drying formulation for the sucrose/mannitol formulation identified in Examples 1 and 2. The sucrose, glycine freeze-drying formulation also showed good reconstitution and stability properties. However, osmolality for this formulation was higher than for the sucrose, mannitol formulation.

Evaluation of buffer composition and buffer pH suggested that a histidine buffer at pH5.5 would be the best choice for a Fab1 freeze-dried formulation.

### Example 4: Long-Term Stability Studies

At increasing pH, there is a risk of acceleration of hydrolysis. In addition, it was shown in example 2 that pH has a dramatic effect on the increase in acidic species. Further, as shown in Example 3, in formulations with 2.0% glycine, and especially in formulations with 2.5% glycine, partial crystallization of glycine was observed. Therefore, the most preferred formulations identified in the previous examples were all assessed at a lower pH, i.e. pH 5.5 instead of pH 6.0 (see Table 10).

TABLE 10
Compositions of the formulations

| | | | Bulking | |
|---|---|---|---|---|
| Formulation | Buffer | Stabilizer | agent | Surfactant |
| I | 20 mM His pH5.5 | 2.5% Suc | 2.5% Man | None |
| J | 20 mM His pH5.5 | 2.5% Suc | 2.5% Man | 0.05% PS20 |
| K | 20 mM His pH5.5 | 2.5% Suc | 2.0% Gly | 0.05% PS20 |

The lyophilizates appeared as an acceptable, white in colour, well-formed cake without defects (pictures not shown). After reconstitution and up to 2 months after the start of the stability studies, the formulations appeared as colourless liquid, slightly opalescent, free of visible particulates. After reconstitution, from 6 to 12 months after the start of the stability studies, the formulations appeared as slightly yellow liquid, slightly opalescent, free of visible particulates.

TABLE 11
Reconstitution time over time

| | T | T = 0 M | T = 1 M | T = 2 M | T = 3 M | T = 6 M | T = 9 M | T = 12 M |
|---|---|---|---|---|---|---|---|---|
| Formulation | (° C.) | (min) | (min) | (min) | (min) | (min) | (min) | (min) |
| I | 5 | 5.6 | 5.9 | 10.2 | 4 | 4 | | |
| | 25 | | 4.9 | 9.3 | 8.5 | 6.2 | | |
| | 30 | | 5.1 | 10.6 | 6.8 | 6.1 | | |
| | 40 | | 8.8 | 10.6 | 4.1 | | | |
| J | 5 | 5.4 | 12.5 | 7.6 | 8.8 | 5 | | |
| | 25 | | 17.4 | 8.8 | 3.7 | 6 | | |
| | 30 | | 12 | 6.4 | 11.2 | 5.4 | | |
| | 40 | | 5.4 | 5.1 | 11.5 | | | |
| K | 5 | 5.7 | 4.2 | 4.3 | 6.6 | 5.1 | 5.2 | 4.5 |
| | 25 | | 4 | 2.5 | 6.5 | 5.3 | 5.1 | 5.7 |
| | 30 | | 5.5 | 4.3 | 7 | 5.5 | | |
| | 40 | | 5.9 | 4.7 | 5.3 | | | |

25° C./60% RH; 30° C./65% RH and 40° C./75% RH

TABLE 12
Aggregation (SE-HPLC) over time

| | | Prelyo | T = 0 | T = 1 M | T = 2 M | T = 3 M | T = 6 M | T = 9 M | T = 12 M |
|---|---|---|---|---|---|---|---|---|---|
| | T | Aggr | Aggr | Aggr | Aggr | Aggr | Aggr | Aggr | Aggr |
| Formulation | (° C.) | (%) | (%) | (%) | (%) | (%) | (%) | (%) | (%) |
| I | 5 | 0.7 | 0.9 | 0.7 | 0.8 | 0.7 | 0.7 | | |
| | 25 | | | 0.8 | 1 | 1.3 | 2.5 | | |
| | 30 | | | 0.9 | 1.5 | 2 | 3.7 | | |
| | 40 | | | 1.3 | 2.5 | 2.9 | | | |
| J | 5 | 0.7 | 0.8 | 0.6 | 0.7 | 0.6 | 0.7 | | |
| | 25 | | | 0.8 | 1.1 | 1.3 | 2.4 | | |
| | 30 | | | 0.9 | 1.5 | 1.9 | 3.8 | | |
| | 40 | | | 1.2 | 2.2 | 3.1 | | | |
| K | 5 | 0.7 | 0.7 | 0.6 | 0.6 | 0.6 | 0.7 | 0.7 | 0.8 |
| | 25 | | | 0.7 | 1 | 1.1 | 2.2 | 2.5 | 3.5 |
| | 30 | | | 0.8 | 1.4 | 1.6 | 3.3 | | |
| | 40 | | | 1.2 | 2.2 | 2.8 | | | |

TABLE 13
Low molecular weight species (SE-HPLC) over time

| | | Prelyo | T = 0 | T = 1 M | T = 2 M | T = 3 M | T = 6 M | T = 9 M | T = 12 M |
|---|---|---|---|---|---|---|---|---|---|
| | T | LMWS | LMWS | LMWS | LMWS | LMWS | LMWS | LMWS | LMWS |
| Formulation | (° C.) | (%) | (%) | (%) | (%) | (%) | (%) | (%) | (%) |
| I | 5 | 0.0 | 0.0 | 0.0 | 0.6 | 0.5 | 0.6 | | |
| | 25 | | | 0.0 | 1.1 | 1.3 | 3.4 | | |
| | 30 | | | 0.0 | 1.3 | 2.2 | 4.9 | | |
| | 40 | | | 0.0 | 1.8 | 2.2 | | | |
| J | 5 | 0.0 | 0.0 | 0.0 | 0.5 | 0.5 | 0.6 | | |
| | 25 | | | 0.0 | 1.1 | 1.6 | 3.5 | | |
| | 30 | | | 0.0 | 1.4 | 2.0 | 5.1 | | |
| | 40 | | | 0.0 | 1.6 | 2.8 | | | |
| K | 5 | 0.0 | 0.0 | 0.0 | 0.4 | 0.5 | 0.6 | 0.1 | N.D |
| | 25 | | | 0.0 | 0.8 | 1.1 | 2.7 | 3.1 | 5.6 |
| | 30 | | | 0.0 | 1.1 | 1.7 | 3.9 | | |
| | 40 | | | | 1.6 | 2.6 | | | |

TABLE 14
Total acidic species over time

| | T | Prelyo | T = 0 | T = 1 M | T = 2 M | T = 3 M | T = 6 M | T = 9 M | T = 12 M |
|---|---|---|---|---|---|---|---|---|---|
| Formulation | (° C.) | (%) | (%) | (%) | (%) | (%) | (%) | (%) | (%) |
| I | 5 | 16.7 | 16.6 | 14.7 | 15 | 15.4 | 17 | | |
| | 25 | | | 15.2 | 16.7 | 17.1 | 18.4 | | |
| | 30 | | | 15.6 | 17.9 | 19.2 | 21.8 | | |
| | 40 | | | 18.4 | 22.5 | 24.2 | | | |
| J | 5 | 16.3 | 16.1 | 14.6 | 15.3 | 15.7 | 17.1 | | |
| | 25 | | | 15.4 | 16.9 | 17.3 | 19 | | |
| | 30 | | | 15.6 | 18.1 | 18.9 | 21.5 | | |
| | 40 | | | 18.5 | 22.5 | 26.2 | 0 | | |
| K | 5 | 16.4 | 16.4 | 15.1 | 15.5 | 16.1 | 16.6 | 13.7 | 14.9 |
| | 25 | | | 15.7 | 17.3 | 18 | 19.5 | 17.7 | 20.1 |
| | 30 | | | 16.1 | 18.6 | 19.8 | 22 | | |
| | 40 | | | 18.9 | 23.5 | 25.1 | 0 | | |

TABLE 15
Total basic species over time

| | T | Prelyo | T = 0 | T = 1 M | T = 2 M | T = 3 M | T = 6 M | T = 9 M | T = 12 M |
|---|---|---|---|---|---|---|---|---|---|
| Formulation | (° C.) | (%) | (%) | (%) | (%) | (%) | (%) | (%) | (%) |
| I | 5 | 1.5 | 1.3 | 4.3 | 4.9 | 4.7 | 0.7 | | |
| | 25 | | | 4.5 | 5.7 | 5.3 | 2.3 | | |
| | 30 | | | 4.2 | 6.7 | 6.7 | 3.7 | | |
| | 40 | | | 5.8 | 9.6 | 9.3 | | | |
| J | 5 | 1.7 | 1.7 | 4.7 | 5 | 4 | 0.7 | | |
| | 25 | | | 4.6 | 6 | 5.6 | 2.7 | | |
| | 30 | | | 5.1 | 7 | 6.4 | 4 | | |
| | 40 | | | 6.1 | 8.5 | 8.8 | | | |
| K | 5 | 1.7 | 1.7 | 4.3 | 4.3 | 3.2 | 0.8 | 2.7 | 4.3 |
| | 25 | | | 4.7 | 5.4 | 4.6 | 2.5 | 5.6 | 11.0 |
| | 30 | | | 5.1 | 6 | 5.7 | 3.5 | | |
| | 40 | | | 5.9 | 8 | 9.3 | | | |

The three formulations were placed on stability study for six months (see Tables 11-15). In view of the six-month-stability data, formulation K was selected and kept on stability for an additional six months. The selected formulation surprisingly showed the most consistent, and shortest, reconstitution time, best stability profile towards aggregation (SE-HPLC and DLS) and good stability towards increase in acidic species (see Tables 11-15).

Formulation I was not selected due to a higher content of large aggregates than polysorbate containing formulations J and K (data not shown). Formulation J was not selected because of the higher variability in reconstitution time compared to formulation K.

### REFERENCES

1. Chang L, et al., 2005, J. Pharm. Sci. 94:1445-55
2. Carpenter et al., 1997, Pharm. Tres. 14:969-975
3. Meyer et al., 2009, Eur. J. Pharma Sci, 38:29-38
4. WO2007124090
5. WO2016128318
6. WO2017194646
7. U.S. Pat. No. 6,372,716
8. WO2019096776
9. Chapman, 2002, Advanced Drug Delivery Reviews, 54, 531-545
10. Liu et al., 2005, AAPS Pharm. Sci. Tech., 6: E150-E157 (internal note: although not cited in the description, need to be cited in the IDS)

### FURTHER ASPECTS OF THE INVENTION

1. A liquid pharmaceutical composition comprising:
   a. a Fab-PEG or Fab′-PEG molecule at a concentration of from about 50 to about 200 mg/mL,
   b. a buffer keeping the pH between about 5.0 to about 7.0,
   c. from about 1 to about 5% w/v of sucrose,
   d. from about 0.5 to about 4% w/v of glycine, and
   e. optionally a surfactant.
2. A freeze-dried formulation obtained by freeze-drying the pharmaceutical composition according to claim 1.
3. A freeze-dried formulation comprising:
   a. from about 50 to about 80% w/w of a Fab-PEG or Fab′-PEG molecule,
   b. from about 3 to about 14% w/w of a buffer keeping the pH between about 5.0 to about 7.0,
   c. from about 7 to about 30% w/w of sucrose,
   d. from about 3.5 to about 24% w/w of glycine, and
   e. optionally a surfactant.
4. The liquid pharmaceutical composition according to claim 1, or the freeze-dried formulation according to claim 2 or claim 3, wherein the Fab-PEG or Fab′-PEG molecule is derived from a humanised or human antibody.
5. The liquid pharmaceutical composition according to claim 1 or claim 4, or the freeze-dried formulation according to any of claims 2 to 4, wherein the Fab-PEG or Fab′-PEG molecule specifically binds to CD40L.
6. The liquid pharmaceutical composition according to any one of claims 1 and 4-5, or the freeze-dried formulation according to any of claims 2 to 5, wherein the Fab-PEG or Fab′-PEG molecule:
   a. comprises CDR-H1 having the sequence as defined in SEQ ID NO:1; CDR-H2 having the sequence as defined in SEQ ID NO:2; CDR-H3 having the sequence as defined in SEQ ID NO:3; CDR-L1 having the sequence as defined in SEQ ID NO:4; CDR-L2 having the sequence as defined in SEQ ID NO:5 and CDR-L3 having the sequence as defined in SEQ ID NO:6; or
   b. comprises a light variable region having the sequence as defined in SEQ ID NO: 7 and a heavy variable region having the sequence as defined in SEQ ID NO: 8; or
   c. comprises a light variable region having at least 80% identity, preferably 90% identity to the sequence as defined in SEQ ID NO: 7 and a heavy variable region having at least 80% identity, preferably 90% identity to the sequence as defined in SEQ ID NO: 8.
7. The liquid pharmaceutical composition or the freeze-dried formulation according to claim 6, wherein the Fab-PEG or Fab′-PEG molecule has
   a) a maleimide group covalently linked to a single thiol group in the modified hinge region; a lysine residue is covalently linked to the maleimide group; and
   b) a methoxypoly(ethyleneglycol) polymer having a molecular weight of approximately 20 KDa is attached to each of the amine groups on the lysine residue.
8. The liquid pharmaceutical composition according to any one of claims 1 and 4-7, or the freeze-dried formulation according to any of claims 2 to 7, wherein the buffer is a histidine buffer.
9. The liquid pharmaceutical composition according to any of claims 1 and 4-8, or the freeze-dried formulation according to any of claims 2 to 8, wherein the concentration of the buffer is at about 10 to about 50 mM.
10. The liquid pharmaceutical composition according to any of claims 1 and 4-8, or the freeze-dried formulation according to any of claims 2 to 8, wherein the surfactant is in an amount of about 0.01 to about 0.2%.
11. The liquid pharmaceutical composition according to any one of claims 1 and 4-10, or the freeze-dried formulation according to any of claims 2 to 10, wherein the optional surfactant is a polysorbate.
12. The liquid pharmaceutical composition or the freeze-dried formulation according to claim 11, wherein the polysorbate is PS20.
13. The liquid pharmaceutical composition according to any one of claims 1 and 4-12, or the freeze-dried formulation according to any of claims 2 to 121, wherein the ratio (w/w) sucrose:glycine is from or from about 2:3 to about 2:1.
14. The liquid pharmaceutical composition according to any one of claims 1 and 4-13, wherein the pharmaceutical composition comprises about 100 mg/mL of the Fab-PEG or Fab′-PEG molecule, about 20 mM of buffer which keep the pH at about 5.5, about 2.5% w/v of sucrose, about 2% w/v of glycine and optionally about 0.05% w/v of PS20.
15. The freeze-dried formulation according to any one of claims 2 to 13, wherein the freeze-dried formulation comprises about 67% w/w of a Fab-PEG or Fab′-PEG molecule, about 2.5-2.6% w/w of a buffer keeping the pH between about 5.0 to about 7.0, about 16.5-16.75% w/w of sucrose, about 13.3-13.5% w/w of glycine, and optionally about 0.3% w/w of a surfactant.
16. A method for producing a freeze-dried formulation according to any of claims 2 to 13 and 15, comprising the steps of:
   a. forming a mixture of the Fab-PEG or Fab′-PEG molecule, together with the buffer, the sucrose, the glycine and the optional surfactant to obtain a pharmaceutical composition and
   b. submitting the mixture of step a. to freeze-drying, and
   c. recovering the freeze-dried formulation.
17. A method for reconstituting the freeze-dried formulation according to any one of claims 2 to 13 and 15 to 16 by adding a solvent, wherein the solvent can be water or a saline buffer.
18. An article of manufacture comprising a container comprising the liquid pharmaceutical composition according to any one of claims 1 and 4-14 or the freeze-dried formulation according to any one of claims 2 to 13 and 15 to 17.

1. A liquid pharmaceutical composition comprising:
a) a Fab-PEG or Fab′-PEG molecule at a concentration of from about 50 to about 200 mg/ml,
b) a buffer keeping the pH between about 5.0 to about 7.0,
c) from about 1 to about 5% w/v of sucrose,
d) from about 0.5 to about 4% w/v of glycine, and
e) optionally a surfactant.

2-18. (canceled)

19. A freeze-dried formulation obtained by freeze-drying the pharmaceutical composition according to claim 1.

20. A freeze-dried formulation comprising:
a) from about 50 to about 80% w/w of a Fab-PEG or Fab′-PEG molecule,
b) from about 2 to about 14% w/w of a buffer keeping the pH between about 5.0 to about 7.0,
c) from about 7 to about 30% w/w of sucrose,
d) from about 3.5 to about 24% w/w of glycine, and
e) optionally a surfactant.

21. The liquid pharmaceutical composition according to claim 1, wherein the Fab-PEG or Fab′-PEG molecule is derived from a humanised or human antibody.

22. The liquid pharmaceutical composition according to claim 21, wherein the Fab-PEG or Fab′-PEG molecule specifically binds to CD40L.

23. The liquid pharmaceutical composition according to claim 21, wherein the Fab-PEG or Fab′-PEG molecule:
a) comprises CDR-H1 having the sequence as defined in SEQ ID NO: 1; CDR-H2 having the sequence as defined in SEQ ID NO: 2; CDR-H3 having the sequence as defined in SEQ ID NO: 3; CDR-L1 having the sequence as defined in SEQ ID NO: 4; CDR-L2 having the sequence as defined in SEQ ID NO: 5 and CDR-L3 having the sequence as defined in SEQ ID NO: 6; or
b) comprises a light variable region having the sequence as defined in SEQ ID NO: 7 and a heavy variable region having the sequence as defined in SEQ ID NO: 8; or
c) comprises a light variable region having at least 80% identity to the sequence as defined in SEQ ID NO: 7 and a heavy variable region having at least 80% identity to the sequence as defined in SEQ ID NO: 8.

24. The liquid pharmaceutical composition according to claim 23, wherein the Fab-PEG or Fab′-PEG molecule has
a) a maleimide group covalently linked to a single thiol group in the modified hinge region; a lysine residue is covalently linked to the maleimide group; and
b) a methoxypoly(ethyleneglycol) polymer having a molecular weight of approximately 20 KDa is attached to each of the amine groups on the lysine residue.

25. The liquid pharmaceutical composition according to claim 1, wherein the buffer is a histidine buffer.

26. The liquid pharmaceutical composition according to claim 1, wherein the concentration of the buffer is at about 10 to about 50 mM.

27. The liquid pharmaceutical composition according to claim 1, wherein the liquid pharmaceutical composition comprises a surfactant present in an amount of about 0.01 to about 0.2% w/v.

28. The liquid pharmaceutical composition according to claim 27, wherein the surfactant is a polysorbate.

29. The liquid pharmaceutical composition according to claim 28, wherein the polysorbate is PS20.

30. The liquid pharmaceutical composition according to claim 1, wherein the ratio (w/w) sucrose:glycine is from about 2:3 to about 2:1.

31. The liquid pharmaceutical composition according to claim 1, wherein the pharmaceutical composition comprises about 100 mg/ml of the Fab-PEG or Fab′-PEG molecule, about 20 mM of a buffer which keeps the pH at about 5.5, about 2.5% w/v of sucrose, about 2% w/v of glycine and optionally about 0.05% w/v of PS20.

32. The freeze-dried formulation according to claim 19, wherein the freeze-dried formulation comprises about 67% w/w of a Fab-PEG or Fab′-PEG molecule, about 2.6-2.8% w/w of a buffer keeping the pH between about 5.0 to about 7.0, about 16.5-16.75% w/w of sucrose, about 13.3-13.5% w/w of glycine, and optionally about 0.3% w/w of a surfactant.

33. A method for producing a freeze-dried formulation comprising the steps of:
a) forming a mixture of the Fab-PEG or Fab′-PEG molecule, together with a buffer keeping the pH between about 5.0 to about 7.0, sucrose, glycine and an optional surfactant to obtain a pharmaceutical composition, and
b) submitting the mixture of step a) to freeze-drying, and
c) recovering the freeze-dried formulation.

34. A method for reconstituting the freeze-dried formulation according to claim 19 comprising adding a solvent to the freeze-dried formulation, wherein the solvent is water or a saline buffer.

35. An article of manufacture comprising a container comprising the liquid pharmaceutical composition according to claim 1.

36. An article of manufacture comprising a container comprising the freeze-dried formulation according to claim 19.


SEQUENCE LISTING (10 sequences)

SEQ ID NO: 1 - protein, 10 aa, synthetic construct
  REGION 1..10: note = CDR-H1
GFSSTNYHVH

SEQ ID NO: 2 - protein, 16 aa, synthetic construct
  REGION 1..16: note = CDR-H2
VIWGDGDTSYNSVLKS

SEQ ID NO: 3 - protein, 10 aa, synthetic construct
  REGION 1..10: note = CDR-H3
QLTHYYVLAA

SEQ ID NO: 4 - protein, 11 aa, synthetic construct
  REGION 1..11: note = CDR-L1
RASEDLYYNLA

SEQ ID NO: 5 - protein, 7 aa, synthetic construct
  REGION 1..7: note = CDR-L2
DTYRLAD

SEQ ID NO: 6 - protein, 9 aa, synthetic construct
  REGION 1..9: note = CDR-L3
QQYYKFPFT

SEQ ID NO: 7 - protein, 107 aa, synthetic construct
  REGION 1..107: note = variable light chain
DIQMTQSPSSLSASVGDRVTITCRASEDLYYNLAWYQRKPGKAPKLLIYDTYRLADGVPSRFSGSGSGTDYTLTISSLQPEDFASYYCQQYYKFPFTFGQGTKVEIK

SEQ ID NO: 8 - protein, 118 aa, synthetic construct
  REGION 1..118: note = variable heavy chain
EVQLVESGGGLVQPGGSLRLSCAVSGFSSTNYHVHWVRQAPGKGLEWMGVIWGDGDTSYNSVLKSRFTISRDTSKNTVYLQMNSLRAEDTAVYYCARQLTHYYVLAAWGQGTLVTVSS

SEQ ID NO: 9 - protein, 214 aa, synthetic construct
  REGION 1..214: note = Fab light chain
DIQMTQSPSSLSASVGDRVTITCRASEDLYYNLAWYQRKPGKAPKLLIYDTYRLADGVPSRFSGSGSGTDYTLTISSLQPEDFASYYCQQYYKFPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 229 aa, synthetic construct
  REGION 1..229: note = Fab heavy chain
EVQLVESGGGLVQPGGSLRLSCAVSGFSSTNYHVHWVRQAPGKGLEWMGVIWGDGDTSYNSVLKSRFTISRDTSKNTVYLQMNSLRAEDTAVYYCARQLTHYYVLAAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA